(19) United States
(12) Patent Application Publication
(10) Pub. No.: US 2001/0007877 A1
(43) Pub. Date: Jul. 12, 2001

(54) NOVEL ANTI-INFECTIVES

(76) Inventors: GEORGE O. BURTON, DOUGLASSVILLE, PA (US); RICHARD M. KEENAN, MALVERN, PA (US); STEVEN D. KNIGHT, WEST CHESTER, PA (US); LANCE H. RIDGERS, COLLEGEVILLE, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 09/437,683

(22) Filed: Nov. 10, 1999

Related U.S. Application Data
(60) Provisional application No. 60/112,482, filed on Dec. 16, 1998.
(60) Provisional application No. 60/112,500, filed on Dec. 16, 1998.
(60) Provisional application No. 60/112,494, filed on Dec. 16, 1998.
(60) Provisional application No. 60/112,463, filed on Dec. 16, 1998.
(60) Provisional application No. 60/112,424, filed on Dec. 16, 1998.
(60) Provisional application No. 60/140,043, filed on Jun. 18, 1999.

(51) Int. Cl. C07D 209/04; A61K 31/4045

(52) U.S. Cl. 514/414; 548/491; 514/415; 514/418

(57) ABSTRACT

Novel anti-infectives and methods of using them are provided.

### FIELD OF THE INVENTION

The present invention relates to novel anti-infectives. Specifically, the present invention involves novel herpesvirus inhibitors specific for the essential interaction between the major capsid protein and the scaffolding protein.

### BACKGROUND OF THE INVENTION

The herpesviruses are a family of large double stranded DNA-containing viruses that include a number of important human pathogens. The herpesvirus family can be divided into three subfamilies: the alphaherpesviruses, betaherpesviruses and gammaherpesviruses. Herpes simplex virus types 1 and 2 (HSV-1, HSV-2) are alphaherpesviruses that cause a wide spectrum of diseases in humans, including keratitis leading to blindness, encephalitis and herpes labialis from HSV-1 infection, and neonatal disease and genital herpes from HSV-2 infection. This subfamily also includes varicella-zoster virus (VZV), the causative agent of chickenpox and shingles. The betaherpesvirus cytomegalovirus (CMV) causes severe clinical disease in the immunosuppressed and immunocompromised populations, including pneumonia, colitis, and retinitis. In addition, congenital CMV infection may cause cytomegalic inclusion disease in babies. The betaherpesvirus human herpesvirus 6 (HHV-6), is the primary cause of roseola (exanthum subitum) in children, and has also been associated with bone marrow graft suppression and interstitial pneumonitis in bone marrow transplant recipients. The betaherpesvirus HHV-7 also causes roseola in a minority of cases, although other disease associations are less clear. The gammaherpesviruses include Epstein Barr virus (EBV), the etiological agent for infectious mononucleosis, Burkitt's lymphoma and nasopharyngeal carcinoma. EBV has also been associated with Hodgkin's disease. The most recently identified gammaherpesvirus, human herpesvirus 8 (HHV-8, also called Kaposi's associated herpesvirus), is believed to be the causative agent of Kaposi's sarcoma and has also been associated with multiple myeloma.

Significant unmet medical need remains within the three herpesvirus subfamilies. With the alphaherpesviruses HSV-1 and 2, current antivirals show only partial efficacy with reductions in pain, lesion severity and shedding. A medical need therefore exists for improvement in overall efficacy, with elimination of pain and decreased viral shedding, lesions, number of recurrences and time to healing. In addition, the ability to impact viral reactivation from latency would be a significant advantage and may have disease-modifying implications. For the betaherpesviruses, current therapies are toxic, primarily intravenous or intravitreal administration. Therefore a key unmet medical need is improved safety over existing therapies, with oral administration and increased potency also important goals. There is no currently approved antiviral therapy for the gammaherpesviruses.

Based on the foregoing, there exists a significant need to identify synthetic or biological compounds for their ability to inhibit herpesviruses.

### SUMMARY OF THE INVENTION

The present invention involves compounds represented by Formula (I) hereinbelow, pharmaceutical compositions comprising such compounds and methods of using the present compounds. The present compounds represent a novel class of anti-herpesvirus inhibitors specific for the essential interaction between the major capsid protein and either the full-length protease or the scaffolding proteins.

### DETAILED DESCRIPTION OF THE INVENTION

The present invention provides compounds of Formula (I), hereinbelow:

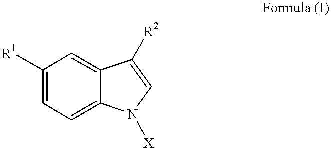

wherein:

R¹ represents ArCH₂O or Ar, wherein Ar represents aryl;

R² represents C₁₋₄NHR, wherein R is H or C(NH)NH₂; and

X represents H or SO₂R, wherein R is selected from the group consisting of C₁₋₂₀ alkyl or aryl.

In one embodiment of the present invention, a compound according to formula (II), hereinbelow:

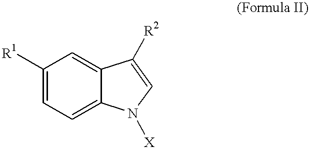

wherein R¹ represents aryl;

R² represents C₁₋₄ NHR, wherein R is H or C(NH)NH₂; and

X represents SO₂R, wherein R is selected from the group consisting of C₁₋₂₀ alkyl or aryl; is provided.

In another alternative embodiment of the present invention, a compound having the structure according to formula (III) hereinbelow:

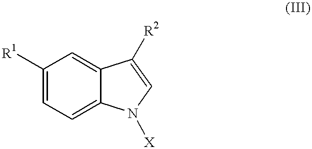

wherein:

R¹ represents ArCH₂O, wherein Ar represents aryl;

R₂ represents (C₁₋₄)NH₂; and

X represents CH₂R, wherein R is selected from the group consisting of C₁₋₂₀ alkyl, aryl and C(O)NR′R″, wherein R′ and R″ are, independently, H or C₁₋₁₀ alkyl or aryl, is provided.

In yet another alternative embodiment of the present invention, a compound according to formula (IV) hereinbelow:

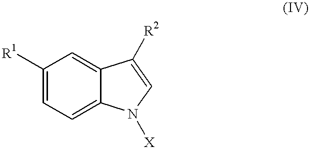

wherein R¹ represents ArCH₂O, wherein Ar represents aryl;

R² represents (C₁₋₄)NH_{2;} and

X represents SO₂R, wherein R is C₁₋₂₀ alkyl or aryl; is provided.

In yet another embodiment of the present invention, a compound according to formula (V) hereinbelow:

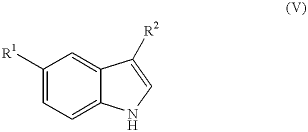

wherein:

R¹ represents aryl; and R² represents (C₁₋₄)NH₂; is provided.

In yet another embodiment of the present invention, a compound according to formula (VI) hereinbelow:

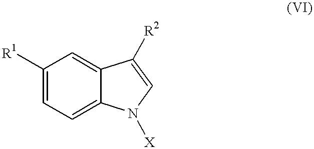

wherein R¹ represents aryl;

R² represents (C₁₋₄)NH₂; and

X represents CH₂R, wherein R represents C₁₋₂₀ alkyl, aryl or C(O)NR′R″, wherein R′ and R″ are, independently, H, C₁₋₁₂ alkyl or C₅₋₁₂ aryl; is provided.

In yet another embodiment of the present invention, a compound according to the formula (VII) hereinbelow:

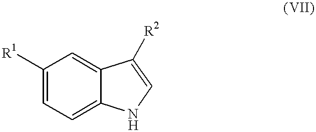

wherein:

R¹ represents ArCH₂O; and

R² represents (C₁₋₄)NH₂; is provided.

Preferably, R represents C₁₋₁₅ alkyl or aryl, more preferably C₁₋₁₀ alkyl or aryl. Preferred aryl substituents at R represent phenyl and naphthyl.

Alternativly, in formula (I), X represents CH₂R, wherein R represents C₁₋₂₀ alkyl, aryl or C(O)NR′R″, wherein R′ and R″ are, independently, H, C₁₋₁₂ alkyl or C₅₋₁₂ aryl.

As used herein, “alkyl” refers to an optionally substituted hydrocarbon group joined together by carbon-carbon bonds. The alkyl hydrocarbon group may be linear, branched or cyclic, saturated or unsaturated. Preferably, the group is saturated, linear or cyclic.

As used herein “aryl” represents an optionally substituted aromatic group with at least one ring having a conjugated pi-electron system. “Aryl” contains 5 to 20 carbon atoms, preferably, 5 to 12. Preferred aryl moieties include naphthyl, dibenzofuranyl, thianaphthyl, benzofuranyl, thienyl, benzophenone, 3-cyanophenyl, 4-cyanophenyl, 4-carboxyphenyl, 3-carboxyphenyl and trifluoromethylphenyl.

The present invention involves the identification of compounds which inhibit the essential interaction between the viral major capsid protein (hereafter referred to as MCP) and the carboxy-terminus of either the full-length protease or the scaffolding proteins. Hereinafter scaffolding protein will be understood to encompass both the full-length protease and the scaffolding proteins in herpesviruses preferably betaherpesviruses most preferably cytomegalovirus (CMV). Deletion analysis has mapped the minimal domain of the scaffolding protein necessary to interact with the major capsid protein to 12 amino acids in HSV-1 and 16 amino acids in CMV. (Hong Z., M. Beaudet-Miller, J. Durkin, R. Zhang, and A. D. Kwong (1996) J. Virol. 70:533-540; Beaudet-Miller M., R. Zhang, J. Durkin, W. Gibson, A. D. Kwong, and Z. Hong. (1996) J. Virol. 70:8081-8088).

### HSV-1 RAADLFVSQMMG

### CMV DMVDLNRRIFVAALNK

wherein:

R represents arginine;

A represents alanine;

D represents aspartic acid;

L represents leucine;

F represents phenylalanine;

V represents valine;

S represents serine;

Q represents glutarmine;

M represents methionine;

G represents glycine;

N represents asparagine;

I represents isoleucine; and

K represents lysine.

Mutational analysis has revealed that the phenylalanine (F) residues, conserved in all herpesviruses identified to date, as well as the hydrophobic nature of the surrounding amino acids, are critical for interaction with the HSV-1 or CMV major capsid protein (Hong Z., M. Beaudet-Miller, J. Durkin, R. Zhang, and A. D. Kwong (1996) J. Virol. 70:533-540; Beaudet-Miller M., R. Zhang, J. Durkin, W. Gibson, A. D. Kwong, and Z. Hong. (1996) J. Virol. 70:8081-8088). This interaction is required for nuclear localization of the major capsid protein and for assembly of the major capsid protein around the scaffold (Wood L. J. et al. 1997.71:179-190, Beaudet-Miller M., R. Zhang, J. Durkin, W. Gibson, A. D. Kwong, and Z. Hong. (1996) J. Virol. 70:8081-8088). As shown in HSV, removal of the carboxy-terminal amino acids of the scaffolding protein prevents interaction with the major capsid protein, thereby abolishing capsid assembly and ultimately inhibiting production of infectious virus (Kennard J., F. J. Rixon, I. M. McDougall, J. D. Tatman and V. G. Preston. (1995) J. Gen. Virol. 76:1611-1621; Thomsen D. T., W. W. Newcomb, J. C. Brown and F. L. Homa. (1995) J. Virol. 69:3690-3703; Matusick-Kumar L., W. W. Newcomb, J. C. Brown, P. J. McCann III, W. Hurlburt, S. P. Weinheimer and M.Gao. (1995) J. Virol. 9:4347-4356; Oien N. L., D. R. Thomsen, M. W. Wathen, W. W. Newcomb, J. C. Brown and F. L. Homa. (1997) J. Virol. 71:1281-1291).

Preferred compounds useful in the present invention include:

1-phenylsulfonyl-3-(2-aminoethyl)-5-(1-naphthyl)indole hydrochloride

1 -(2-naphthylsulfonyl)-3-(2-guanidinoethyl)-5-(1-naphthyl)indole hydrochloride

1-phenylsulfonyl-3-(2-aminoethyl)-5-(2-thienyl)indole hydrochloride

1-phenylsulfonyl-3-(2-aminoethyl)-5-(3-trifluoromethylphenyl)indole hydrochloride; and

1-[(2-naphthyl)sulfonyl]-3-(2-aminoethyl)-5-(1-naphthyl )indole hydrochloride

1-phenylsulfonyl-3-(2-aminoethyl)-5-(2-naphthyl)indole hydrochloride

1-phenylsulfonyl-3-(2-aminoethyl)-5-phenylindole hydrochloride

1-phenylsulfonyl-3-(2-aminoethyl)-5-(benzothiophen-2-yl)indole hydrochloride

1-phenylsulfonyl-3-aminomethyl-5-(1-naphthyl)indole trifluoroacetate

4-[1-phenylsulfonyl-3-(2-aminoethyl)indol-5-yl]benzophenone

2-[1-Benzenesulfonyl-5-(4-methyl-naphthalen-1-yl)-1-H-indol-3-yl]-ethylainine

4-[3-(2-Amino-ethyl)-1-benzenesulfonyl-1-H-indol-5-yl]-naphthalen-1-ylamine

2-[1-Benzenesulfonyl-5-(2-methoxymethyl-naphthalen-1-yl)-1-H-indol-3-yl]-ethylamine

6-[3-(2-Amino-ethyl)-1-benzenesulfonyl-1-H-indol-5-yl]-naphthalen-2-ol

1-[(3-carbamoylphenyl)methyl]-3-(2-aminoethyl)-5-(4-dibenzofuranyl)indole hydrochloride;

1-(5-cyanopentyl)-3-(2-aminoethyl)-5-(4-dibenzofuranyl)indole hydrochloride;

1-(2-carbamoylethyl)-3-(2-aminoethyl)-5-(4-dibenzofuranyl)indole hydrochloride;

1-[(3-carboxyphenyl)methyl]-3-(2-aminoethyl)-5-(1-naphthyl)indole hydrochloride; and

3-(2-aminoethyl)-5-(4-dibenzofuranyl)indole-1-pentanoic acid hydrochloride

1-[(3-carbamoylphenyl)methyl]-3-(2-aminoethyl)-5-(4-dibenzofuranyl)indole hydrochloride;

1-(5-cyanopentyl)-3-(2-aminoethyl)-5-(4-dibenzofuranyl)indole hydrochloride;

1-(2-carbamoylethyl)-3-(2-aminoethyl)-5-(4-dibenzofuranyl)indole hydrochloride;

1-[(3-carboxyphenyl)methyl]-3-(2-aminoethyl)-5-(1-naphthyl)indole hydrochloride; and

3-(2-aminoethyl)-5-(4-dibenzofuranyl)indole-1-pentanoic acid hydrochloride

1-(carbamoylmethyl)-3-(2-aminoethyl)-5-(2-naphthyl)indole trifluoroacetate

1-(N-methylcarbamoylmethyl)-3-(2-aminoethyl)-5-(1-naphthyl)indole trifluoroacetate

1-[(3-carbamoylphenyl)methyl]-3-(2-aminoethyl)-5-(1-naphthyl)indole hydrochloride

1-[(3-morpholinoylphenyl)methyl]-3-(2-aminoethyl)-5-(1-naphthyl)indole hydrochloride

1-[(3-(3-trifluoromethylbenzyl)carbamoylphenyl)methyl]-3-(2-aminoethyl)-5-(1-naphthyl)indole hydrochloride

1-[(4-carbamoylphenyl)methyl]-3-(2-aminoethyl)-5-(4-dibenzofuranyl)indole hydrochloride

1-(carbamoylmethyl)-3-(2-aminoethyl)-5-(4-dibenzofuranyl)indole hydrochloride

1-(4-carbomethoxyphenyl)methyl-3-(2-aminoethyl)-5-(4-dibenzofuranyl)indole hydrochloride

1-(3-carbomethoxyphenyl)methyl-3-(2-aminoethyl)-5-(4-dibenzofuranyl)indole hydrochloride

1-(2-cyanophenyl)methyl-3-(2-aminoethyl)-5-(4-dibenzofuranyl)indole hydrochloride

1-(3-cyanophenyl)methyl-3-(2-arminoethyl)-5-(4-dibenzofuranyl)indole hydrochloride

1-(4-acetamidophenyl)methyl-3-(2-aminoethyl)-5-(4-dibenzofuranyl)indole hydrochloride

1-(4-cyanobutyl)-3-(2-aminoethyl)-5-(4-dibenzofuranyl)indole hydrochloride

1-[(2-carbamoylphenyl)methyl]-3-(2-aminoethyl)-5-(4-dibenzofuranyl)indole hydrochloride

1-ethyl-3-(2-aminoethyl)-5-(4-dibenzofuranyl)indole hydrochloride

1-[(3-carboxyphenyl)methyl]-3-(2-aminoethyl)-(5-(benzothiophen-2-yl)indole hydrochloride

1-[(4-carboxyphenyl)methyl]-3-(2-aminoethyl)-5-(benzothiophen-2-yl)indole hydrochloride

1-[(3-carboxyphenyl)methyl]-3-(2-aminoethyl)-5-(4-dibenzofuranyl)indole hydrochloride

3-(2-aminoethyl)-5-(4-dibenzofuranyl)indole-1-butanoic acid hydrochloride

1-[(N-(3-trifluoromethylphenyl)methyl)acetamido]-3-(2-aminoethyl)-5-(1-naphthyl)indole

1-[(N-(4-pyridyl)methyl)acetamido]-3-(2-aminoethyl)-5-(1-naphthyl)indole

1-[(N-(3-methoxyphenyl)methyl)acetamido]-3-(2-aminoethyl)-5-(1-naphthyl)indole

1-[(N-(4-sulfonamidophenyl)methyl)acetamido]-3-(2-aminoethyl)-5-(1-naphthyl)indole

1-[(N-benzyl)acetamido]-3-(2-aminoethyl)-5-(1-naphthyl)indole

1-[(N-(2,5-difluorophenyl)methyl)acetamido]-3-(2-aminoethyl)-5-(1-naphthyl)indole

1-[(N-(2,4-dichlorophenyl)methyl)acetamido]-3-(2-aminoethyl)-5-(1-naphthyl)indole

1-[(N-(2-benzimidazole)methyl)acetamido]-3-(2-aminoethyl)-5-(1-naphthyl)indole

1-[N-(3-pyridyl)acetamido]-3-(2-aminoethyl)-5-(1-naphthyl)indole

1-[N-(2-thiazole)acetamido]-3-(2-aminoethyl)-5-(1-naphthyl)indole

1-[(N-(2-thiophene)methyl)acetamido]-3-(2-aminoethyl)-5-(1-naphthyl)indole

1-[trans-(N-cyclopropylphenyl)acetamido]-3-(2-aminoetbyl)-5-(1-naphthyl)indole

1-[(N-(4-carboethoxy)piperidine)acetamido]-3-(2-aminoethyl)-5-(1-naphthyl)indole

1-[N-(3-methoxyphenyl)acetamido]-3-(2-aminoethyl)-5-(4-dibenzofuranyl)indole.

3-(2-aminoethyl)-5-(2-naphthyl)indole hydrochloride

3-(2-aminoethyl)-5-(1-naphthyl)indole hydrochloride

3-(2-aminoethyl)-5-(benzofuran-2-yl)indole hydrochloride

3-(3-aminopropyl)-5-(1-naphthyl)indole trifluoroacetate; and

3-(2-aminoethyl)-5-(4-dibenzofuranyl)indole hydrochloride

3-(2-aminoetbyl)-5-(2-thienyl)indole hydrochloride

3-(2-aminoethyl)-5-(3-trifluoromethylphenyl)indole hydrochloride

4-[3-(2-aminoethyl)indol-5-yl]benzophenone hydrochloride

3-(2-aminoethyl)-5-(benzothiophen-2-yl)indole hydrochloride

2-[5-(4-Methyl-naphthalen-1-yl)-1 H-indol-3-yl]-ethylamine

6-[3-(2-Amino-ethyl)-1 H-indol-5-yl]-naphthalen-2-ol

7-[3-(2-Amino-ethyl)-1 H-indol-5-yl]-3-hydroxy-naphthalene-2-carboxylic acid (2-methoxy-phenyl)-amide

2-[5-(6-Methoxy-naphthalen-2-yl)-1 H-indol-3-yl]-ethylamine

1-(4-methoxy)benzenesulfonyl-3-(2-aminoethyl)-5-(1-naphthyl)methyloxyindole trifluoroacetate,

1-(4-methoxy)benzenesulfonyl-3-(2-aminoethyl)-5-(2-naphthyl)methyloxyindole trifluoroacetate,

1-(8-quinoline)sulfonyl-3-(2-aminoethyl)-5-(2-naphthyl)methyloxyindole trifluoroacetate,

1-(2-thienyl)sulfonyl-3-(2-aminoethyl)-5-(4-biphenyl)methyloxyindole trifluoroacetate, and

1-(2-chloro-4-fluoro)benzenesulfonyl-3-(2-aminoethyl)-5-(2-biphenyl)methyloxyindole trifluoroacetate

1-(3-chloro-4-fluoro)benzenesulfonyl-3-(2-aminoethyl)-5-(2-naphthyl)methyloxyindole trifluoroacetate

1-(8-quinoline)sulfonyl-3-(2-aminoethyl)-5-(2-biphenyl)methyloxyindole trifluoroacetate

1-(4-methoxy)benzenesulfonyl-3-(2-aminoethyl)-5-(4-biphenyl)methyloxyindole trifluoroacetate

1-(2-thienyl)sulfonyl-3-(2-aminoethyl)-5-[(4-tert-butyl)phenyl] methyloxyindole trifluoroacetate

3-(2-aminoethyl)-5-[(3-phenoxy)benzyloxy]indole hydrochloride,

3-(2-aminoethyl)-5-[(2-naphthyl)methyloxy]indole hydrochloride,

3-(2-aminoethyl)-5-[(2-phenyl)benzyloxy]indole hydrochloride, and

3-(2-aminoethyl)-5-[(4-phenyl)benzyloxy]indole hydrochloride

1-(N-Carboxmethyl-N-methylcarbamoylmethyl)-3-(2-aminoethyl)-5-(4-dibenzofuranyl)indole trifluoroacetate

3-[3-(2-Amino-ethyl)-5-(4-cyano-benzyloxy)-indol-1-ylmethyl]-benzoic acid trifluoroacetate

({2-[3-(2-Amino-ethyl)-5-(3-cyano-benzyloxy)-indol-1-yl]-ethanoyl}-methyl-amino)-acetic acid

3-[3-(2-Amino-ethyl)-5-(4-carbamoyl-benzyloxy)-indol-1-ylmethyl]-benzoic acid

3-[3-(2-Amino-ethyl)-5-(4-carboxy-benzyloxy)-indol-1-ylmethyl]-benzoic acid

3-[3-(2-Amino-ethyl)-5-(3-carboxy-benzyloxy)-indol-1-ylmethyl]-benzoic acid

[3-(2-Amino-ethyl)-5-(4-carbamoyl-benzyloxy)-indol-1-yl]-acetic acid

4-[3-(2-Amino-ethyl)-1-carboxymethyl-1 H-indol-5-yloxymethyl]-benzoic acid

[3-(2-Amino-ethyl)-5-(3-carbamoyl-benzyloxy)-indol-1-yl]-acetic acid

3-[3-(2-Amino-ethyl)-1-carboxymethyl-1H-indol-5-yloxymethyl]-benzoic acid

4-[3-(2-Amino-ethyl)-5-(3-carboxy-benzyloxy)-indol-1-ylmethyl]-benzoic acid

4-[3-(2-Amino-ethyl)-1-(3-cyano-benzyl)-1H-indol-5-yloxymethyl]-benzoic acid

5-[3-(2-Amino-ethyl)-5-(3-carbamoyl-benzyloxy)-indol-1-ylmethyl]-furan-2-carboxylic acid

5-[3-(2-Amino-ethyl)-5-(3-cyano-benzyloxy)-indol-1-ylmethyl]-furan-2-carboxylic acid

4-[3-(2-Amino-ethyl)-5-(4-carbamoyl-benzyloxy)-indol-1-ylmethyl]-benzoic acid

5-[3-(2-Amino-ethyl)-5-(4-carbamoyl-benzyloxy)-indol-1-ylmethyl]-furan-2-carboxylic acid

[3-(2-Amino-ethyl)-5-(4-cyano-benzyloxy)-indol-1-yl]-acetic acid

5-[3-(2-Amino-ethyl)-5-(4-cyano-benzyloxy)-indol-1-ylmethyl]-furan-2-carboxylic acid

4-[3-(2-Amino-ethyl)-5-(3-carbamoyl-benzyloxy)-indol-1-ylmethyl]-benzoic acid

3-[3-(2-Amino-ethyl)-5-(3-carbamoyl-benzyloxy)-indol-1-ylmethyl]-benzoic acid

5-[3-(2-Amino-ethyl)-5-(3-carbamoyl-benzyloxy)-indol-1-ylmethyl]-thiophene-2-carboxylic acid

3-[3-(2-Amino-ethyl)-5-(3-cyano-benzyloxy)-indol-1-ylmethyl]-benzoic acid

5-[3-(2-Amino-ethyl)-5-(3-cyano-benzyloxy)-indol-1-ylmethyl]-thiophene-2-carboxylic acid

4-[3-(2-Amino-ethyl)-5-(biphenyl-4-ylmethoxy)-indol-1-ylmethyl]-benzoic acid

3-[3-(2-Amino-ethyl)-5-(biphenyl-4-ylmethoxy)-indol-1-ylmethyl]-benzoic acid

5-[3-(2-Amino-ethyl)-5-(biphenyl-4-ylmethoxy)-indol-1-ylmethyl]-furan-2-carboxylic acid

6-[3-(2-Amino-ethyl)-5-(2-chloro-4-fluoro-benzyloxy)-indol-1-ylmethyl]-nicotinic acid

5-[3-(2-Amino-ethyl)-5-(2-chloro-4-fluoro-benzyloxy)-indol-1-ylmethyl]-thiophene-2-carboxylic acid

3-[3-(2-Amino-ethyl)-5-(2-chloro-4-fluoro-benzyloxy)-indol-1-ylmethyl]-benzoic acid

5-[3-(2-Amino-ethyl)-5-(2-chloro-4-fluoro-benzyloxy)-indol-1-ylmethyl]-furan-2-carboxylic acid

4-[3-(2-Amino-ethyl)-5-(2-chloro-4-fluoro-benzyloxy)-indol-1-ylmethyl]-benzoic acid

(S)-1-{2-[3-(2-Amino-ethyl)-5-(2-chloro-4-fluoro-benzyloxy)-indol-1-yl]-ethanoyl}-pyrrolidine-2-carboxylic acid

6-[3-(2-Amino-ethyl)-5-(biphenyl-2-ylmethoxy)-indol-1-ylmethyl]-nicotinic acid

5-[3-(2-Amino-ethyl)-5-(biphenyl-2-ylmethoxy)-indol-1-ylmethyl]-thiophene-2-carboxylic acid

3-[3-(2-Amino-ethyl)-5-(biphenyl-2-ylmethoxy)-indol-1-ylmethyl]-benzoic acid

5-[3-(2-Amino-ethyl)-5-(biphenyl-2-ylmethoxy)-indol-1-ylmethyl]-furan-2-carboxylic acid

4-[3-(2-Amino-ethyl)-5-(biphenyl-2-ylmethoxy)-indol-1-ylmethyl]-benzoic acid

6-[3-(2-Amino-ethyl)-5-(2-benzyloxy-ethoxy)-indol-1-ylmethyl]-nicotinic acid

5-[3-(2-Amino-ethyl)-5-(2-benzyloxy-ethoxy)-indol-1-ylmethyl]-thiophene-2-carboxylic acid

3-[3-(2-Amino-ethyl)-5-(2-benzyloxy-ethoxy)-indol-1-ylmethyl]-benzoic acid

5-[3-(2-Amino-ethyl)-5-(2-benzyloxy-ethoxy)-indol-1-ylmethyl]-furan-2-carboxylic acid

4-[3-(2-Amino-ethyl)-5-(2-benzyloxy-ethoxy)-indol-1-ylmethyl]-benzoic acid

3-[3-(2-Amino-ethyl)-5-(2-fluoro-benzyloxy)-indol-1-ylmethyl]-benzoic acid

5-[3-(2-Amino-ethyl)-5-(2-fluoro-benzyloxy)-indol-1-ylmethyl]-furan-2-carboxylic acid

4-[3-(2-Amino-ethyl)-5-(2-fluoro-benzyloxy)-indol-1-ylmethyl]-benzoic acid

2-[3-(2-Amino-ethyl)-5-(2-fluoro-benzyloxy)-indol-1-ylmethyl]-5-bromo-benzoic acid

5-[3-(2-Amino-ethyl)-5-(3-nitro-benzyloxy)-indol-1-ylmethyl]-thiophene-2-carboxylic acid

3-[3-(2-Amino-ethyl)-5-(3-nitro-benzyloxy)-indol-1-ylmethyl]-benzoic acid

5-[3-(2-Amino-ethyl)-5-(3-nitro-benzyloxy)-indol-1-ylmethyl]-furan-2-carboxylic acid

6-[3-(2-Amino-ethyl)-5-(3-cyano-benzyloxy)-indol-1-ylmethyl]-nicotinic acid

1-{2-[3-(2-Amino-ethyl)-5-(3-cyano-benzyloxy)-indol-1-yl]-ethanoyl}-pyrrolidine-2-carboxylic acid

4-[3-(2-Amino-ethyl)-5-(6-chloro-benzo[1,3]dioxol-5-ylmethoxy)-indol-1-ylmethyl]-benzoic acid

6-[3-(2-Amino-ethyl)-5-(3-methoxy-benzyloxy)-indol-1-ylmethyl]-nicotinic acid

5-[3-(2-Amino-ethyl)-5-(3-methoxy-benzyloxy)-indol-1-ylmethyl]-furan-2-carboxylic acid

4-[3-(2-Amino-ethyl)-5-(3-methoxy-benzyloxy)-indol-1-ylmethyl]-benzoic acid

6-[3-(2-Amino-ethyl)-5-(6-fluoro-4H-benzo[1,3]dioxin-8-ylmethoxy)-indol-1-ylmethyl]-nicotinic acid

5-[3-(2-Amino-ethyl)-5-(6-fluoro-4H-benzo[1,3]dioxin-8-ylmethoxy)-indol-1-ylmethyl]-thiophene-2-carboxylic acid

3-[3-(2-Amino-ethyl)-5-(6-fluoro-4H-benzo[1,3]dioxin-8-ylmethoxy)-indol-1-ylmethyl]-benzoic acid

5-[3-(2-Amino-ethyl)-5-(6-fluoro-4H-benzo[1,3]dioxin-8-ylmethoxy)-indol-1-ylmethyl]-furan-2-carboxylic acid

4-[3-(2-Amino-ethyl)-5-(6-fluoro-4H-benzo[1,3]dioxin-8-ylmethoxy)-indol-1-ylmethyl]-benzoic acid

2-[3-(2-Amino-ethyl)-5-(6-fluoro-4H-benzo[1,3]dioxin-8-ylmethoxy)-indol-1-ylmethyl]-5-bromo-benzoic acid

1-{2-[3-(2-Amino-ethyl)-5-(6-fluoro-4H-benzo[1,3]dioxin-8-ylmethoxy)-indol-1-yl]-ethanoyl}-pyrrolidine-2-carboxylic acid

5-[3-(2-Amino-ethyl)-5-(3-cyano-benzyloxy)-indol-1-ylmethyl]-thiophene-2-carboxylic acid

3-[3-(2-Amino-ethyl)-5-(3-cyano-benzyloxy)-indol-1-ylmethyl]-benzoic acid

6-[3-(2-Amino-ethyl)-5-(3-nitro-benzyloxy)-indol-1-ylmethyl]-nicotinic acid

2-[3-(2-Amino-ethyl)-5-(3-nitro-benzyloxy)-indol-1-ylmethyl]-5-bromo-benzoic acid

(S)-1-{2-[3-(2-Amino-ethyl)-5-benzyloxy-indol-1-yl]-ethanoyl}-pyrrolidine-2-carboxylic acid

2-[3-(2-Amino-ethyl)-5-(3-cyano-benzyloxy)-indol-1-ylmethyl]-5-bromo-benzoic acid

5-[3-(2-Amino-ethyl)-5-(6-chloro-benzo[1,3]dioxol-5-ylmethoxy)-indol-1-ylmethyl]-thiophene-2-carboxyic acid

3-[3-(2-Amino-ethyl)-5-(3-methoxy-benzyloxy)-indol-1-ylmethyl]-benzoic acid

2-[3-(2-Amino-ethyl)-5-(6-chloro-benzo[1,3]dioxol-5-ylmethoxy)-indol-1-ylmethyl]-5-bromo-benzoic acid

5-[3-(2-Amino-ethyl)-5-benzyloxy-indol-1-ylmethyl]-furan-2-carboxylic acid

5-[3-(2-Amino-ethyl)-5-(3-carbamoyl-benzyloxy)-indol-1-ylmethyl]-2-bromo-benzoic acid

3-[3-(2-Amino-ethyl)-5-(6-chloro-benzo[1,3]dioxol-5-ylmethoxy)-indol-1-ylmethyl]-benzoic acid

5-[3-(2-Amino-ethyl)-5-(6-chloro-benzo[1,3]dioxol-5-ylmethoxy)-indol-1-ylmethyl]-furan-2-carboxylic acid

1-(4-trifluoromethylphenyl)methyl-3-(2-aminoethyl)-5-(2-naphthyl)methyloxyindole trifluoroacetate,

1-(3-trifluoromethylphenyl)methyl-3-(2-aminoethyl)-5-(4-cyanophenyl)methyloxyindole trifluoroacetate,

1-(3,4-dichlorophenyl)methyl-3-(2-aminoethyl)-5-(4-cyanophenyl)methyloxyindole trifluoroacetate,

1-(3,4-dichlorophenyl)methyl-3-(2-aminoethyl)-5-(3-cyanophenyl)methyloxyindole trifluoroacetate, and

1-[(3,5-bis-trifluoromethyl)phenyl]methyl-3-(2-aminoethyl)-5-(3-cyanophenyl)methyloxyindole trifluoroacetate

1-[(3,5-bis-trifluoromethyl)phenyl]methyl-3-(2-aminoethyl)-5-(4-cyanophenyl)methyloxyindole trifluoroacetate

1-(4-trifluoromethylphenyl)methyl-3-(2-aminoethyl)-5-(3-cyanophenyl)methyloxyindole trifluoroacetate.

The compounds of the present invention may contain one or more asymmetric carbon atoms and may exist in racemic and optically active forms. All of these compounds and diastereomers are considered to be within the scope of the present invention.

The present compounds can also be formulated as pharmaceutically acceptable salts and complexes thereof. Pharmaceutically acceptable salts are non-toxic salts in the amounts and concentrations at which they are administered.

Pharmaceutically acceptable salts include acid addition salts such as those containing sulfate, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present.

The present compounds can be prepared by the processes exemplified hereinbelow:

1) 5-Bromotryptamine (1) was protected as its tert-butoxycarbonyl urethane and subsequently treated with PhSO₂Cl to provide sulfonamide 2. Sulfonamide 2 was treated with 1-naphthylboronic acid under standard Suzuki reaction conditions to give 3, see Miyaura, N.; Suzuki, A Chem. Rev. 1995, 95, 2457, which was subsequently treated with 4 N HCl in dioxane to furnish the target compound 4. Alternativly, removal of the sulfonyl group from 3, followed by treatment of 5 with 4 N HCl in dioxane provided the target compound 6.

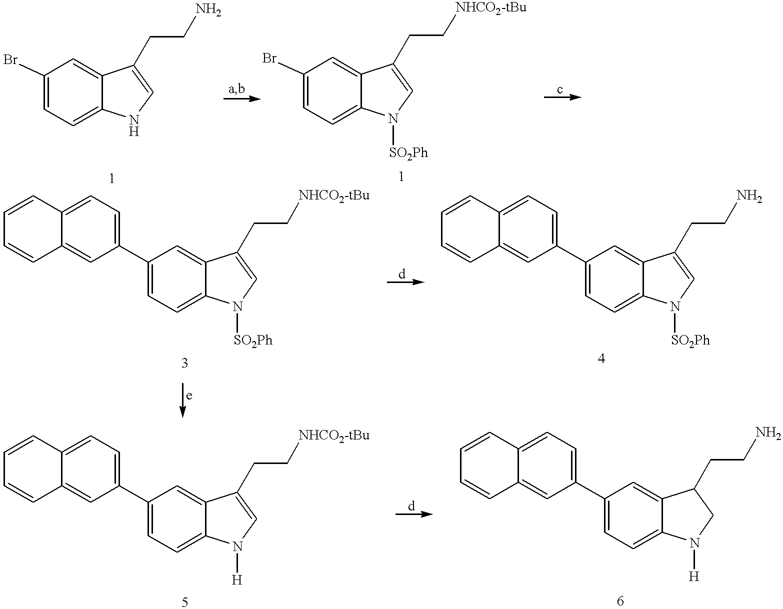

Conditions: a) Di-t-butyl dicarbonate, Et₃N, CH₂Cl₂; b) PhSO₂Cl, NaOH, Bu₄NHSO₄, CH₂Cl₂; c) 2-naphthylboronic acid, Pd(PPh₃)₄, Cs₂CO₃, DME/H₂O; d) 4 N HCl/dioxane e) K₂CO₃, MeOH/H₂O, reflux.

2) Selective alkylation of the 5-hydroxyl group of N-tert-butoxycarbonyl serotonin (7) gave ether 8. Alkylation with 4-trifluoromethylbenzyl bromide to furnish 9, followed by treatment with 4 N HCl in dioxane provided the target compound 10.

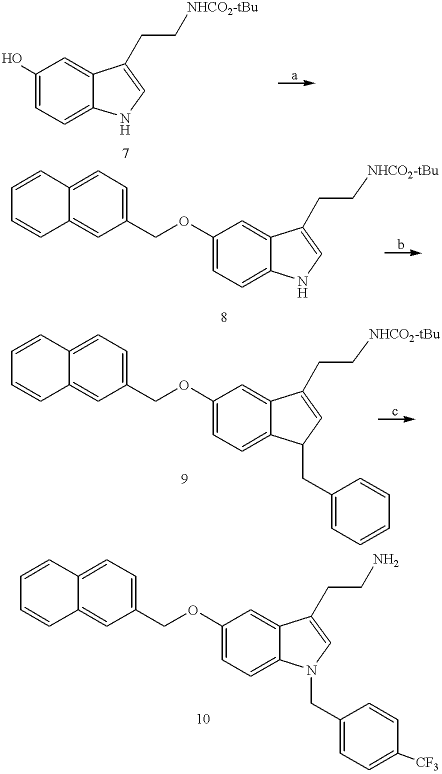

Conditions: a) 2-Bromomethylnaphthylene, K₂CO₃, KI, acetone; b) 4-trifluoromethylbenzyl bromide, NaH, DMF; c) 4 N HCl/dioxane.

3) Sulfonylation of the indole nitrogen of ether 8 to provide 11, followed by treatment with 4 N HCl in dioxane furnished the target compound 12.

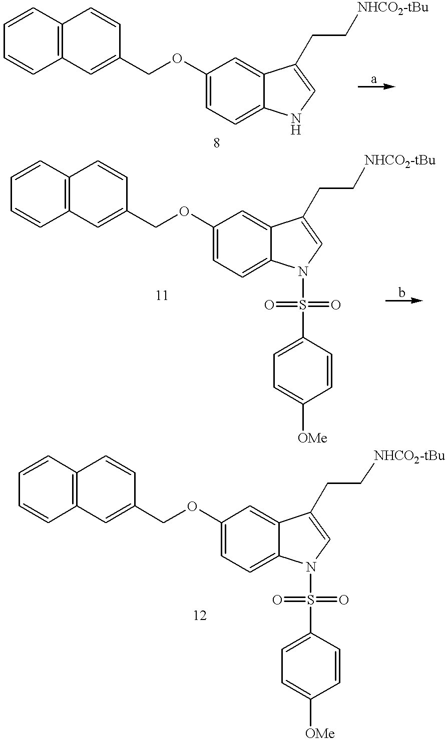

Conditions: a) 4-methoxybenzenesulfonyl chloride, NaOH, Bu₄NHSO₄, CH₂Cl₂; b) 4 N HCl/dioxane.

4) O-Alkylation of N-tert-butoxycarbonyl serotonin (7) under standard Mitsunobu reaction conditions yielded ether 13. Treatment of 13 with 4 N HCl in dioxane provided the target compound 14.

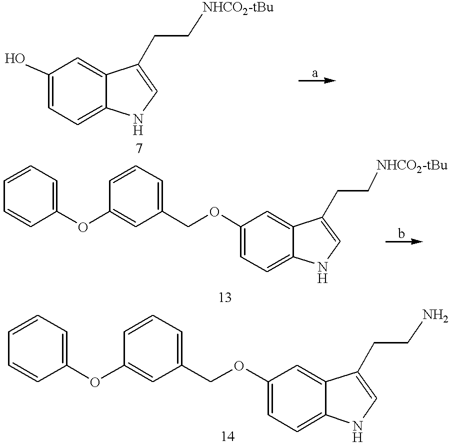

Conditions: a) 3-Phenoxybenzyl alcohol, diisopropyl azodicarboxylate, PPh₃, CH₂Cl₂; b) 4 N HCl/dioxane.

5) Alkylation of the indole nitrogen of 5 under standard conditions yielded 15. Treatment of 15 with 4 N HCl in dioxane provided a target compound 16, and hydrolysis of the ester function under standard conditions provides a further target compound 17.

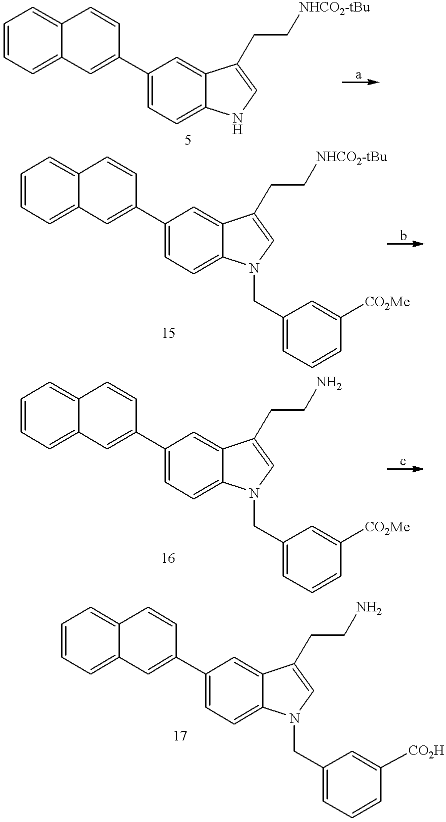

Conditions: a) methyl 4-bromomethylbenzoate, Cs₂CO₃, DMA; b) 4 N HCl/dioxane, CH₂Cl₂; c) 1N NaOH, MeOH.

With appropriate manipulation and protection of any chemical functionality, synthesis of the remaining compounds of Formula (1) is accomplished by methods analogous to those above.

In order to use a compound of Formula (I) or a pharmaceutically acceptable salt thereof for the treatment of humans and other mammals, it is normally formulated in accordance with standard pharmaceutical practice as a pharmaceutical composition.

The present ligands can be administered by different routes including intravenous, intraperitoneal, subcutaneous, intramuscular, oral, topical, transdermal, or transmucosal administration. For systemic administration, oral administration is preferred. For oral administration, for example, the compounds can be formulated into conventional oral dosage forms such as capsules, tablets and liquid preparations such as syrups, elixirs and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art. The amounts of various compounds to be administered can be determined by standard procedures taking into account factors such as the compound (IC₅₀) potency, (EC₅₀) efficacy, and the biological half-life (of the compound), the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered. Oral administration is a preferred method of administration of the present compounds.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula(I). A topical formulation contains suitably 0.01 to 5.0% of a compound of Formula (I). The active ingredient may be administered from 1 to 6 times per day, preferably once, sufficient to exhibit the desired activity, as is readily apparent to one skilled in the art.

As used herein, “treatment” of a disease includes, but is not limited to prevention, retardation, prophylaxis, therapy and cure of the disease. As used herein, “diseases” treatable using the present compounds include, but are not limited to keratitis, encephalitis, herpes labialis, neonatal disease, genital herpes, chicken pox, shingles, pneumonia, colitis, retinitis, cytomegalic inclusion disease, roseola, febrile seizures, bone marrow graft suppression, interstitial pneumonitis, multiple sclerosis, mononucleosis, Burkitt's lymphoma, nasopharyngeal carcinoma, Hodgkin's disease, Kaposi's sarcoma, and multiple myeloma.

Composition of Formula (I) and their pharmaceutically acceptable salts which are active when given orally can be formulated as syrups, tablets, capsules and lozenges. A syrup formulation will generally consist of a suspension or solution of the compound or salt in a liquid carrier for example, ethanol, peanut oil. olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

No unacceptable toxological effects are expected when compounds of the present invention are administered in accordance with the present invention.

The biological activity of the compounds of Formula (I) are demonstrated by the tests indicated hereinbelow.

Examples were tested employing the assay conditions described below and gave an IC₅₀ (concentration at which binding is reduced by 50%) of 1-10 uM. ELISA assay for detection of inhibitors of the interaction between the CMV MCP full-length protein and the interaction domain peptide of the scaffolding protein.

Inhibitor activity was detected using a competitive enzyme-linked immunosorbant assay (ELISA) that utilizes time resolved fluorometry. In this assay, the scaffolding protein peptide and the compound were added simultaneously to a microtiter plate precoated with full-length MCP. A primary antibody specific for the scaffolding protein-peptide is then added, followed by a Europium (Eu)-labeled secondary antibody, directed against the primary antibody and fluorescence activity measured. If the compound is unable to compete for binding, then the peptide binds to MCP and a fluorescent signal is detected. If the compound inhibits the binding of the scaffolding protein peptide to MCP, then the primary antibody cannot bind and a reduction in fluorescence is observed. In all assays a background control is included in which no peptide is added, but all other reagents are identical. In addition, a set of samples in which no compound is added is also included. This value is set at 100% and the percent inhibition is calculated relative to this number.

Compounds with an IC₅₀ (concentration at which binding is reduced by 50%), of less than 10 uM were the preferred compounds. Compounds with an IC₅₀ greater than 50 uM were considered inactive.

The detailed procedure for the ELISA was as follows. It is not intended to be limiting in any way. A skilled artisan can readily modify the same for other applications within the scope of the present invention. The procedure detailed is also applicable to all other herpesviruses.

1. High-binding microtiter plates (Costar-384 well high binding microtiter plates) were coated with 50 ul per well of a 1/50 dilution of recombinant baculovirus supernatant containing MCP (see below for generation method) diluted in 50 mM carbonate-bicarbonate buffer pH 9.6 (Sigma; St Louis, Mo.). The plates were incubated at room temperature for two hours or 4° C. overnight.

2. The plates were washed 4-5 times with 100 ul per well of 1X PBS (137 mM NaCl, 8 mM Na₂HPO₄, 2.68 mM KCl, 1.47 mM KH₂PO₄, pH 7.0)+0.02% Triton X-100, followed by incubation with 0.25 ug of scaffolding protein peptide (25 mer; see below for sequence) diluted in 50 ul PBS for with increasing concentrations of compound (2-fold dilutions from 50 uM to 0.048 uM), for 1 hour at room temperature.

3. The plates were then washed to remove any unbound peptide as in step 2, followed by the addition of 100 ul per well of 5% BSA diluted in PBS for 1 hour at room temperature to block nonspecific interactions.

4. The plates were washed as before, and 50 ul per well of a primary antipeptide antibody raised against a carboxy-teminal 25-mer peptide diluted 1/5000 in 10X PBS was added and incubated for 1 hour at room temperature.

5. The plates were washed as before to remove any free antibody, and then incubated with 50 ul per well (2.5 ng/ml) of secondary antibody (Eu-labeled anti-rabbit IgG; (Wallac; Gaithersburg, Md.); diluted in IX PBS with 5% BSA and 0.02% Triton X-100, and incubated at room temperature for 1 hour.

6. The plates were washed 6 times as in step 2, and 50 ul per well of Wallac enhancement solution (Wallac; Gaithersburg, Md) added, and the plate shaken for 10 minutes at room temperature, before being read at on a Wallac VICTOR microtiter plate reader (Wallac; Gaithersburg, Md.).

Generation of reagents: Full-length MCP, scaffolding protein peptide, primary anti-peptide antibody

a) Full-length MCP protein.

Fall armyworm Spodoptera frugiperda (SF9) cells (American Type Culture Collection Manassas, Va.) were maintained in Sf-900 II SFM media (Gibco BRL, Life Technologies) supplemented with 5% (v/v) heat inactivated fetal bovine serum (FBS; HyClone Laboratories, Inc). Recombinant baculovirus Autographa californiica nuclear polyhedrosis virus expressing full-length CMV MCP) was propagated and grown as described (Ausubel F. M. et al., Current Protocols in Molecular Biology). In order to generate protein supernatants, SF9 cells were seeded in T175 tissue culture flasks and incubated at 28° C. Cells were infected at a multiplicity of 1 plaque forming unit per cell, in a low volume of tissue culture medium. After 1 hour, additional media was added and the infected cells were harvested 72 to 96 hours post-infection.

b.) Peptide synthesis. A linear peptide containing the scaffolding protein minimal interaction domain were synthesized by California Peptide Research, Inc. (Napa, Calif.) or American Peptide Company, (Sunnyvale, Calif.).

Amino Acid Sequence of peptide used in ELISA assay:

CMV: NH₂-Ala-Ser-Gln-Ser-Pro-Pro-Lys-Asp-Met-Val-Asp-Leu-Asn-Arg-Arg-Ile-Phe-Val-Ala-Ala-Leu-Asn-Lys-Leu-Glu-COOH

c.) Primary antipeptide antibody:

Polyclonal antipeptide antibodies were generated by Covance Research Products Inc. (Denver, Pa.) to the following peptide which contains the portion of UL80.5 necessary for the interaction with the MCP.

CMV 25-mer peptide with the following sequence:

NH₂-Ala-Ser-Gln-Ser-Pro-Pro-Lys-Asp-Met-Val-Asp-Leu-Asn-Arg-Arg-Ile-Phe-Val-Ala-Ala-Leu-Asn-Lys-Leu-Glu-COOH

wherein:

Ala represents alanine;

Ser represents serine;

Gln represents glutamine;

Pro represents proline;

Lys represents lysine;

Asp represents aspartic acid;

Met represents methionine;

Val represents valine;

Asn represents asparagine;

Arg represents arginine;

Ile represents isoleucine;

Phe represents phenylalanine; and

Glu represents glutamic acid.

The present invention includes but is not limited to the following examples:

### EXAMPLE 1

### Preparation of 1-phenylsulfonyl-3-(2-aminoethyl)-5-(2-naphthyl)indole hydrochloride

a) 1-phenylsulfonyl-3-[2-(tert-butoxycarbonylamino)ethyl]-5-(2-naphthyl)indole

A solution of 3-[2-(tert-butoxycarbonylamino)ethyl]-5-bromoindole (2.35 g, 6.93 mmol) and tetrabutylammonium hydrogen sulfate (0.35 g, 1.04 mmol) in methylene chloride (50 mL) was cooled to 0° C. and treated with several portions of freshly powdered NaOH (5.54 g, 139 mmol). The slurry was stirred at this temperature for 15 min, then treated with benzenesulfonyl chloride (7.1 mL, 55.4 mmol) in methylene chloride (25 mL) over 5 min. The mixture stirred at 0° C. for 45 min, then at room temperature overnight. It was diluted with methylene chloride (50 mL) and filtered. The filtered solid was rinsed with methylene chloride (2×25 mL) and the filtrate evaporated to an orange oil. The crude product was purified by flash chromatography (silica gel, 25% ethyl acetate/hexane) and carried on to the next reaction.

A solution of the purified 1-phenylsulfonyl-3-[2-(tert-butoxycarbonyl-amino)ethyl]-5-bromoindole (0.43 g, 0.90 mmol) and cesium carbonate (1.2 g, 3.6 mmol) in ethylene glycol dimethyl ether (25 mL) and water (1 mL) was degassed with argon for 10 min. The solution was treated with Pd(Ph₃P)₄ (0.03 g, 0.03 mmol) followed by 2-naphthaleneboronic acid (0.17 g, 1.01 mmol). Argon was bubbled through for an additional 5 min at room temperature and for 20 h at reflux. The mixture was cooled to room temperature and diluted with 10% sodium hydroxide (20 mL), saturated sodium chloride (20 mL), and diethyl ether (20 mL). The organic layer was separated, dried over Na₂SO₄, and concentrated to a brown oil which was purified by flash chromatography (silica gel, 33% ethyl acetate/hexane) to give the product (0.19 g, 22%) as a white solid. ¹H NMR (CDCl₃)-? 8.11-8.03 (2H, m), 7.93-7.85 (5H, m), 7.78-7.67 (3H, m), 7.56-7.43 (6H, m), 4.70-4.55 (1H, m), 3.48-3.46 (2H, m), 2.94 (2H, t, J=6.8 Hz), 1.42 (9H, s).

b) 1-phenylsulfonyl-3-(2-aminoethyl)-5-(2-naphthyl)indole hydrochloride

The compound of Example 1(a) (0.03 g, 0.08 mmol) was taken up in diethyl ether (10 mL) and treated with 4 N HCl in dioxane (1 mL). The slurry stirred for 1 h and was filtered. The gray solid product (0.12 g, 44%) was rinsed with diethyl ether and vacuum dried over potassium hydroxide. MS (ES+) m/e 427 [M+H]⁺, 468.

### EXAMPLE 2

### Preparation of 3-(2-aminoethvl)-5-(2-naphthvl)indole hydrochloride

a) 3-[2-(tert-butoxycarbonylamino)ethyl]-5-(2-naphthyl)indole

The compound of Example 1(a) (0.19 g, 0.36 mmol) and potassium carbonate (1.0 g, 7.2 mmol) were taken up in 5:1 methanol/water (25 mL). The slurry was heated at reflux for 20 h, then cooled and concentrated. The residue was dissolved in ethyl acetate and washed with water (10 mL) and saturated sodium chloride (10 mL). It was dried over Na₂SO₄ and concentrated to furnish the product (0.12 g, 86%) as a colorless resin. ¹H NMR (CDCl₃)-δ8.12-8.08 (2H, m), 7.93-7.82 (5H, m), 7.60 (1H, dd, J=8.5 Hz, J=1.7 Hz), 7.54-7.44 (3H, m), 7.10 (11H, d, J=1.9 Hz), 4.75-4.60 (11H, m), 3.54-3.52 (2H, m), 3.03 (2H, t, J=6.7 Hz), 1.42 (9H, s)

b) 3-(2-aminoethyl)-5-(2-naphthyl)indole hydrochloride

The compound of Example 2(a) (0.03 g, 0.08 mmol) was taken up in diethyl ether (10 mL) and treated with 4 N HCl in dioxane (1 mL). The slurry stirred for 1 h and was filtered. The solid product (0.12 g, 44%) was rinsed with diethyl ether and vacuum dried over potassium hydroxide. MS (ES+) m/e 287 [M+H]⁺, 328

### EXAMPLE 3

### Preparation of 1-phenylsulfonyl-3-(2-aminoethyl)-5-(1-naphthyl)indole hydrochloride

Following the procedure of Example 1(a) and 1(b), except substituting 1-naphthaleneboronic acid for 2-naphthaleneboronic acid, the title compound was prepared as a yellow solid. MS (ES+) m/e 427 [M+H]⁺, 468

### EXAMPLE 4

### Preparation of 3-(2-aminoethyl)-5-(1-naphthyl)indole hydrochloride

Following the procedure of Example 2(a) and 2(b), except substituting 1-phenylsulfonyl-3-[2-(tert-butoxycarbonylamino)ethyl]-5-(1-naphthyl)indole from Example 3, the title compound was prepared as a yellow solid. MS (ES+) m/e 287 [M+H]⁺, 328

### EXAMPLE 5

### Preparation of 1-phenylsulfonyl-3-(2-aminoethyl)-5-(2-thienyl)indole hydrochloride

Following the procedure of Example 1(a) and 1(b), except substituting 2-thienyl-boronic acid for 2-naphthaleneboronic acid, the title compound was prepared as a yellow solid. MS (ES+) m/e 382 [M]⁺, 423

### EXAMPLE 6

### Preparation of 3-(2-aminomethyl)-5-(2-thienyl)indole hydrochloride

Following the procedure of Example 2(a) and 2(b), except substituting 1-phenylsulfonyl-3-[2-(tert-butoxycarbonylamino)ethyl]-5-(2-thienyl)indole from Example 5, the title compound was prepared as a yellow solid. MS (ES+) m/e 243 [M+H]⁺, 284

### EXAMPLE 7

### Preparation of 3-(2-aminoethyl)-5-(benzofuran-2-yl)indole hydrochloride

Following the procedure of Example 1(a) then 2(a) and 2(b) except substituting benzofuran-2-ylboronic acid for 2-naphthaleneboronic acid, the title compound was prepared as a brown powder. MS (ES+) m/e 277 [M+H]⁺, 318

### EXAMPLE 8

### Preparation of 1-phenylsulfonyl-3-(2-aminoethyl)-5-(3-trifluoromethylphenyl)indole hydrochloride

Following the procedure of Example 1(a) and 1(b), except substituting 3-trifluoromethylphenylboronic acid for 2-naphthaleneboronic acid, the title compound was prepared as an off-white powder. MS (ES+) m/e 445 [M+H]⁺, 486

### EXAMPLE 9

### Preparation of 3-(2-aminoethyl)-5-(3-trifluoromethylphenyl)indole hydrochloride

Following the procedure of Example 2(a) and 2(b), except substituting 1-phenylsulfonyl-3-[2-(tert-butoxycarbonylamino)ethyl]-5-(3-trifluoromethylphenyl)indole from Example 8, the title compound was prepared as an off-white solid. MS (ES+) m/e 305 [M+H]⁺, 346

### EXAMPLE 10

### Preparation of 1-phenylsulfonyl-3-(2-aminoethyl)-5-(4-dibenzofuranyl)indole hydrochloride

Following the procedure of Example 1(a) and 1(b), except substituting 4-dibenzofuranylboronic acid for 2-naphthaleneboronic acid, the title compound was prepared as a yellow solid. MS (ES+) m/e 467 [M+H]⁺, 508

### EXAMPLE 11

### Preparation of 3-(2-aminoethyl)-5-(4-dibenzofuranyl)indole hydrochloride

Following the procedure of Example 2(a) and 2(b), except substituting 1-phenylsulfonyl-3-[2-(tert-butoxycarbonylamino)ethyl]-5-(4-dibenzofuranyl)indole from Example 10, the title compound was prepared as a yellow solid. MS (ES+) m/e 327 [M+H]⁺, 368

### EXAMPLE 12

### Preparation of 3-(3-aminopropyl)-5-(1-naphthyl)indole trifluoroacetate

a) 3-(2-cyanoethylenyl)-5-(1-naphthyl)indole

A solution of 5-bromoindole-3-carboxaldehyde (0.30 g, 1.3 mmol) and cesium carbonate (2.2 g, 6.7 mmol) in 25:3 ethylene glycol dimethyl ether/water (50 mL) was degassed with argon for 10 min. Pd(PPh₃)₄ (0.08 g, 0.07 mmol) was added and the solution was degassed for an additional 5 min. 1-Naphthyleneboronic acid (0.46 g, 2.7 mmol) was then added and the reaction heated to reflux for 4 h. The mixture was diluted with diethyl ether (20 mL) and washed 2×10% sodium hydroxidelbrine (1:1, 20 mL) and 1×brine (20 mL). It was dried over MgSO₄ and concentrated to a crude yellow solid. This solid was taken up in 1:1 tetrahydrofuran/dioxane (7 mL) and heated at 70° C. until all solid was dissolved. (Cyanomethyl)triphenylphosphorane (0.44 g, 1.5 mmol) was added and the reaction maintained at 70° C. for 1.5 h, then stirred at 48° C. for 2.5 d. It was cooled to room temperature, diluted with diethyl ether (20 mL) and washed 2×5% KHSO₄ (20 mL) and 1×brine (20 mL). It was dried over MgSO₄, concentrated, and purified by flash chromatography (silica gel, step gradient, 20%-30% ethyl acetate/hexane) to furnish the product (0.11 g, 40%) as a yellow oil. ¹H NMR (DMSO) ? 12.01 (1H, s), 8.09-7.20 (12H, m), 6.09 (1H, d, J=16.7 Hz)

b) 3-(3-aminopropyl)-5-(1-naphthyl)indole trifluoroacetate

The compound of Example 7(a) (0.11 g, 0.37 mmol) was dissolved in methanol/ethyl acetate (7 mL). 5% Pd/C (11 mg, 10 wt %) was added, and the mixture shook under hydrogen (50 psi) for 20 h. Additional Pd/C (11 mg, 10 wt %) was added and shaking under hydrogen continued for another 24 h. The mixture was filtered through Celite, concentrated, and purified by preparative HPLC to give the title compound (0.012 g, 11% yield) as a gray solid. MS (ES+) m/e 301 [M+H]⁺, 342

### EXAMPLE 13

### Preparation of 1-phenylsulfonyl-3-(2-aminoethyl)-5-phenylindole hydrochloride

Following the procedure of Example 1(a) and 1(b), except substituting phenylboronic acid for 2-naphthaleneboronic acid, the title compound was prepared as a yellow solid. MS (ES+) m/e 377 [M+H]⁺, 418

### EXAMPLE 14

### Preparation of 4-[1-phenylsulfonyl-3-(2-aminoethyl)indol-5-yl]benzophenone

a) 4-[1-phenylsulfonyl-3-(2-(tert-butoxycarbonylamino)ethyl)indol-5-yl]benzophenone

A flask containing 4-bromobenzophenone (0.26 g, 1.00 mmol), diboron pinacol ester (0.28 g, 1.10 mmol), PdCl₂(dppf).CH₂Cl₂ (0.025g, 0.03 mmol), and acetic acid, potassium salt (0.30 g, 3.01 mmol) was flushed with argon and diluted with dry dimethylformamide (6 mL). The mixture was heated to 80° C. under argon where it stirred for 1.5 h. The solution was allowed to cool to room temperature, at which time 1-phenylsulfonyl-3-[ 2-(tert-butoxycarbonyl-amino)ethyl]-5-bromoindole (0.24 g, 0.50 mmol), PdCl₂(dppf).CH₂Cl₂ (0.025 g, 0.03 mmol), and cesium carbonate (1.63 g, 5.00 mmol) in water (2 mL) were added. The mixture was heated to 80° C. where it stirred for 3 h. It was cooled to room temperature, diluted with saturated sodium bicarbonate (10 mL), and extracted with diethyl ether (10 mL). The organic layer was washed with water (10 mL) and brine (10 mL), dried over MgSO₄, filtered, and concentrated to a yellow residue. Purification by flash chromatography (silica gel, step gradient, 25-33% ethyl acetate/hexane) furnished the product (0.13 g, 45%) as a colorless resin.

b) 4-[1-phenylsulfonyl-3-(2-aminoethyl)indol-5-yl]benzophenone

Following the procedure of Example 1(b), except using 4-[ 1-phenylsulfonyl-3-(2-(tert-butoxycarbonylamino)ethyl)indol-5-yl]benzophenone in place of 1-phenylsulfonyl-3-[ 2-(tert-butoxycarbonylamino)ethyl]-5-(2-naphthyl)indole, the title compound was prepared as a pink solid. MS (ES+) m/e 481 [M+H]⁺, 522

### EXAMPLE 15

### Preparation of 4-[3-(2-aminoethyl)indol-5-yl]benzophenone hydrochloride

a) 4-[3-(2-(tert-butoxycarbonylamino)ethyl)indol-5-yl]benzophenone

A solution of the compound of Example 14(a) (0.12 g, 0.21 mmol) and potassium carbonate (0.58 g, 4.20 mmol) in 5:1 methanol/water (25 mL) was heated at reflux for 20 h. The mixture was cooled to room temperature and concentrated to a crude residue which was partitioned between ethyl acetate (20 mL) and water (20 ML). The organic layer was separated and washed with water (20 mL), saturated sodium bicarbonate (20 mL), and brine (20 mL). It was dried over Na₂SO₄, filtered, and concentrated to give the product (0.09 g, 95%) as a colorless resin.

b) 4-[3-(2-aminoethyl)indol-5-yl]benzophenone hydrochloride

Following the procedure of 2(b), except substituting the compound of Example 15(a), the title compound was prepared (0.05 g, 72%) as a gray powder. MS (ES+) m/e 341 [M+H]⁺, 382

### EXAMPLE 16

### Preparation of 1-phenylsulfonyl-3-(2-aminoethyl)-5-(benzothiophen-2-yl)indole hydrochloride

Following the procedure of Example 1(a) and 1(b), except substituting benzo[6] thiophene-2-boronic acid for 2-naphthaleneboronic acid, the title compound was prepared as a tan solid. MS (ES+) m/e 433 [M+H]⁺, 474

### EXAMPLE 17

### Preparation of 3-(2-aminoethyl)-5-(benzothiophen-2-yl)indole hydrochloride

Following the procedure of Example 2(a) and 2(b), except substituting 1-phenylsulfonyl-3-[2-(tert-butoxycarbonylamino)ethyl]-5-(benzothiophen-2-yl)indole from Example 15, the title compound was prepared as a tan solid. MS (ES+) m/e 293 [M+H]⁺, 334

### EXAMPLE 18

### Preparation of 1-[(2-naphthyl)sulfonyl]-3-(2-aminoethyl)-5-(1-naphthyl)indole hydrochloride

Following the procedure of Example 1(a) and 1(b), except substituting 2-naphthylsulfonyl chloride for benzenesulfonyl chloride and 1-naphthaleneboronic acid for 2-naphthaleneboronic acid, the title compound was prepared as a yellow solid. MS (ES+) m/e 477 [M+H]⁺, 518

### EXAMPLE 19

### Preparation of 1-phenylsulfonyl-3-aminomethyl-5-(1-naphthyl)indole trifluoroacetate

a) 3-(tert-butyloxycarbonylaminomethyl)-5-bromoindole

A solution of 5-bromoindole-3-carboxaldehyde (0.59 g, 2.64 mmol) and ammonium acetate (2.03 g, 26.4 mmol) in methanol (20 mL) was treated with sodium cyanoborohydride (0.33 g, 5.28 mmol) in several portions. The mixture was stirred at room temperature for 2 h, then heated to reflux where it stirred for 5 h. The reaction was cooled to room temperature and concentrated to an oily residue which was dissolved in ethyl acetate (10 mL) and washed with water (10 mL), saturated sodium bicarbonate (10 mL) and brine (10 mL). The organic layer was dried over Na₂SO₄, filtered, and concentrated to furnish the crude product as a yellow oil.

A solution of the crude amine (0.54 g, 2.42 mmol) and triethylamine (0.84 mL, 6.04 mmol) in methylene chloride (25 mL) was cooled to 0° C. The mixture was treated with BOC₂O (0.53 g, 2.42 mmol) and allowed to stir while warming slowly to room temperature. After 72 h, the reaction was quenched by addition of 1 N HCl (10 mL). The organic layer was separated and the aqueous layer extracted with methylene chloride (10 mL). The combined organic layers were washed with 1 N HCl (2×20 mL) and brine (1×20 mL), dried over Na₂SO₄, filtered, and concentrated. The crude product was purified by flash chromatography (silica gel, 33% ethyl acetate/hexane) to give the product (0.13 g, 15%) as a pale yellow solid.

b) 1-phenylsulfonyl-3-(tert-butyloxycarbonylaminomethyl)-5-bromoindole

A solution of the compound of Example 19(a) (0.12 g, 0.37 mmol) and tetrabutylammonium hydrogen sulfate (0.02 g, 0.06 mmol) in methylene chloride (15 mL) was cooled to 0° C. The mixture was treated with several portions of powdered NaOH (0.30 g, 7.44 mmol) and was allowed to stir at 0° C. for 15 min. A solution of benzenesulfonyl chloride (0.38 mL, 3.0 mmol) in methylene chloride (5 mL) was then added. The reaction was allowed to warm to room temperature while stirring for 24 h. The solids were filtered and rinsed with methylene chloride. The filtrate was stirred over saturated sodium bicarbonate (25 mL) for 30 min and the organic layer separated. The organic layer was washed with brine (25 mL), dried over Na₂SO₄, filtered, and concentrated to furnish the clean product (0.17 g, 99%) as a yellow resin.

c) 1-phenylsulfonyl-3-(tert-butyloxycarbonylaminomethyl)-5-(1-naphthyl)indole

A solution of the compound of Example 19(b) (0.17 g, 0.37 mmol) and cesium carbonate (0.60 g, 1,85 mmol) in 25:3 ethylene glycol dimethyl ether/water (28 mL) was degassed with argon for 15 min. Pd(PPh₃)₄ was added, and the mixture was degassed with argon for an additional 5 min. 1-Naphthaleneboronic acid was then added, and the mixture heated to reflux where it stirred under argon for 24 h. The reaction was cooled to room temperature and quenched by addition of 10% sodium hydroxide (20 mL), brine (20 mL), and diethyl ether (40 mL). The organic layer was washed with brine (25 mL), dried over Na₂SO₄, filtered, and concentrated. The crude product was purified by flash chromatography (silica gel, step gradient, 25-33% ethyl acetate/hexane) to give the product (0.14 g, 74%) as a colorless resin.

d) 1-phenylsulfonyl-3-aminomethyl-5-(1-naphthyl)indole trifluoroacetate

The compound of Example 19(c) (0.59 g, 0.12 mmol) was dissolved in 1:1 trifluoroacetic acid/methylene chloride (3 mL) at 0° C. The solution was allowed to warm to room temperature while stirring for 45 min. It was concentrated to an oil which was taken up into anhydrous diethyl ether (5 mL). The salt began to precipitate, so the slurry was stirred at room temperature for 1 h. The precipitated product was filtered and rinsed with diethyl ether to give the title compound (0.20 g, 33%) as a white solid. MS (ES+) m/e 396 [M−NH₃]⁺, 454

### EXAMPLE 20

### Preparation of 1-(2-naphthylsulfonyl)-3-(2-guanidinoethyl)-5-(1-naphthyl)indole hydrochloride

The compound of Example 18 (0.06 g, 0.11 mmol) was dissolved in ethanol (15 mL). 3,5-Dimethylpyrazole-1-carboxamidine (0.023 g, 0.11 mmol) was added, followed by triethylamine (0.016 mL, 0.11 mmol). The mixture was heated to reflux and allowed to stir at this temperature for 24 h. Additional 3,5-dimethylpyrazole-1-carboxamidine (0.023, 0.11 mmol) was added and reflux continued for another 24 h. The mixture was concentrated to an oil and purified by preparative HPLC (step gradient, 10-90% acetonitrile/water+0.1% trifluoroacetic acid, 15 min run) to furnish the title compound (0.011 g, 17%) as a white solid. MS (ES+) m/e 519 [M+H]⁺, 560.

### EXAMPLE 21

### Preparation of 1-(4-trifluoromethylphenyl)methyl-3-(2-aminoethyl)-5-(2-naphthyl)methyloxyindole trifluoroacetate

To a solution of 3-[2-(tert-butoxycarbonylamino)ethyl]-5-hydroxyindole (0.25 g, 0.9 mmol) in acetone (10 mL) was added 2-bromomethylnaphthalene (0.22 g, 1.0 mmol), potassium carbonate (0.38 g, 2.7 mmol), and potassium iodide (0.015 g, 0.09 mmol). The mixture was allowed to stir at room temperature for 5 d, then was filtered through Celite and concentrated. The residue was dissolved in dimethylformamide (3 mL), and sodium hydride (0.01 g, 0.4 mmol) was added. The mixture was allowed to stir at room temperature for 30 min, at which point 0.25M 4-trifluoromethylbenzyl bromide in dimethylformamide (1 mL) was added. The mixture stirred at room temperature for 16 h, and Argonaut's PS-thiophenol resin was added to remove excess 4-trifluoromethylbenzyl bromide. After 2 d of additional stirring, the mixture was filtered and concentrated. The residue was diluted with diethyl ether (5 mL) and treated with 4 N HCl in dioxane (2 mL). The solution stirred at room temperature for 2 h and was concentrated. The residue was purified by preparative HPLC (gradient, 10-90% acetonitrile/water+0.1% trifluoroacetic acid, 15 min run) to furnish the title compound. MS (ES+) m/e 475 [M+H]⁺, 493

### EXAMPLE 22

### Preparation of 1-(3-trifluoromethylphenyl)methyl-3-(2-aminoethyl)-5-(4-cyanophenyl) methyloxyindole trifluoroacetate

Following the procedure of Example 21, except substituting 4-cyanobenzyl bromide for 2-bromomethylnaphthalene and 3-trifluoromethylbenzyl bromide for 4-trifluoromethyl-benzyl bromide, the title compound was prepared. MS (ES+) m/e 450 [M+H]⁺, 472

### EXAMPLE 23

### Preparation of 1-[(3,5-bis-trifluoromethyl)phenyl]methyl-3-(2-aminoethyl)-5-(4-cyanophenyl)methyloxyindole trifluoroacetate

Following the procedure of Example 21, except substituting 4-cyanobenzyl bromide for 2-bromomethylnaphthalene and 3,5-bis-trifluoromethylbenzyl bromide for 4-trifluoromethylbenzyl bromide, the title compound was prepared. MS (ES+) m/e 518 [M+H]⁺, 1035

### EXAMPLE 24

### Preparation of 1-(3,4-dichlorophenyl)methyl-3-(2-aminoethyl)-5-(4-cyanophenyl) methyloxyindole trifluoroacetate

Following the procedure of Example 21, except substituting 4-cyanobenzyl bromide for 2-bromomethylnaphthalene and 3,4-dichlorobenzyl bromide for 4-trifluoromethylbenzyl bromide, the title compound was prepared. MS (ES+) m/e 450 [M]⁺, 452 [M+2]⁺, 454 [M+4]⁺, 901

### EXAMPLE 25

### Preparation of 1-(4-trifluoromethylphenyl)methyl-3-(2-aminoethyl)-5-(3-cyanophenyl) methyloxyindole trifluoroacetate

Following the procedure for Example 21, except substituting 3-cyanobenzyl bromide for 2-bromomethylnaphthalene, the title compound was prepared. MS (ES+) mne 450 [M+H]⁺, 491

### EXAMPLE 26

### Preparation of 1-[(3,5-bis-trifluoromethyl)phenyl]methyl-3-(2-aminoethyl)-5-(3-cyanophenyl)methyloxyindole trifluoroacetate

Following the procedure of Example 21, except substituting 3-cyanobenzyl bromide for 2-bromomethylnaphthalene and 3,5-bis-trifluoromethylbenzyl bromide for 4-trifluoro-methylbenzyl bromide, the title compound was prepared. MS (ES+) m/e 518 [M+H]⁺, 559

### EXAMPLE 27

### Preparation of 1-(3,4-dichlorophenyl)methyl-3-(2-aminoethyl)-5-(3-cyanophenyl) methyloxyindole trifluoroacetate

Following the procedure of Example 21, except substituting 3-cyanophenylbenzyl bromide for 2-bromomethylnaphthalene and 3,4-dichlorobenzyl bromide for 4-trifluoro-methylbenzyl bromide, the title compound was prepared. MS (ES+) m/e 450 [M]⁺, 452 [M+2]⁺

### EXAMPLE 28 (METHOD A)

### Preparation of 1-(4-carboxyphenyl)methyl-3-(2-aminoethyl)-5-(3-cyanophenyl) methyloxyindole trifluoroacetate

Following the procedure of Example 21, except substituting 3-cyanophenylbenzyl bromide for 2-bromomethylnaphthalene and methyl 4-bromobenzoate for 4-trifluoro-methylbenzyl bromide, the title compound was prepared. MS (ES+) m/e 426 [M+H]⁺, 467

### EXAMPLE 28 (METHOD B)

### Preparation of 1-(4-carboxyphenyl)methyl-3-(2-aminoethyl)-5-(3-cyanophenyl) methyloxyindole trifluoroacetate

a) N-t-Butoxycarbonyl-O-3-cyanobenzyl-serotonin

N-t-Butoxycarbonyl-serotonin (540 mg, 1.95 mmol), 3-cyanobenzyl bromide (570 mg, 2.9 mmol) potassium iodide (32 mg, 0.2 mmol) and powdered potassium carbonate (680 mg, 4.9 mmol) in acetone (20 ml, butan-2-one may be substituted for acetone) were heated under reflux for 24 h. The cooled mixture was filtered and the filtrate concentrated then flash chromatographed on silica gel eluting with ethyl acetate in hexane (step gradient, 20-50%) to yield the title compound (604 mg, 79%).

b) N-t-Butoxycarbonyl-O-cyanobenzyl-1-(4-methoxycarbonylbenzyl)-serotonin.

Powdered cesium carbonate (54 mg, 0.17 mmol) was added to a solution of N-t-butoxycarbonyl-O-3-cyanobenzylserotonin (65 mg, 0.17 mmol) and methyl 4-bromomethylbenzoate (48 mg, 0.21 mmol) in N,N-dimethylacetamide (1 ml). The mixture was stirred at RT for 48 h, filtered and the filtrate diluted with water, extracted twice with ethyl acetate. The combined extracts were washed with brine, dried, concentrated and flash chromatographed eluting with ethyl acetate in hexane (step gradient 20-40%) to give the title compound (76 mg, 85%).

c) O-3-Cyanobenzyl-1-(4-methoxycarbonylbenzyl)serotonin.

N-t-Butoxycarbonyl-O-3-cyanobenzyl-1-(4-metboxycarbonylbenzyl)-serotonin (76 mg) was dissolved in 4N hydrogen chloride in dioxan (1 ml) and set aside at RT for 1 h. The solution was evaporated to dryness to provide the title compound as a colourless crystalline solid (68 mg) MS (ES+) m/e 440 [M+H]⁺, 481.

d) 1-(4-Carboxylbenzyl)-O-3-cyanobenzylserotonin

2.5N Aqueous sodium hydroxide (1 ml) was added to O-3-Cyanobenzyl-1-(4-methoxycarbonyl-benzyl)serotonin (68 mg) in methanol (2 ml) and stirred for 2.5 h at RT. The reaction was quenched with hydrochloric acid, concentrated in vacuo, diluted with DMSO and the title compound isolated by preperative HPLC (gradient, 10-90% acetonitrile/water+0.1% trifluoroacetic acid) (29 mg, 48%) MS (ES+) m/e 426 [M+H]⁺, 467.

### EXAMPLE 28 (METHOD C)

### Preparation of 1-(4-carboxyphenyl)methyl-3-(2-aminoethyl)-5-(3-cyanophenyl) methyloxyindole trifluoroacetate

a) N-t-Butoxycarbonyl-O-3-cyanobenzyl-serotonin

N-t-Butoxycarbonyl-serotonin (540 mg, 1.95 mmol), 3-cyanobenzyl bromide (570 mg, 2.9 mmol) potassium iodide (32 mg, 0.2 mmol) and powdered potassium carbonate (680 mg, 4.9 mmol) in acetone (20 ml, butan-2-one may be substituted for acetone) were heated under reflux for 24 h. The cooled mixture was filtered and the filtrate concentrated then flash chromatographed on silica gel eluting with ethyl acetate in hexane (step gradient, 20-50%) to yield the title compound (604 mg, 79%)

b) N-t-Butoxycarbonyl-O-3-cyanobenzyl-1-(4-methoxycarbonylbenzyl)-serotonin. A 1M solution of potassium t-butoxide in t-butanol (0.375 ml, 0.375 mmol) was added to N-t-butoxycarbonyl-O-3-cyanobenzylserotonin (0.125 mmol) in N,N-dimethylformamide (1 ml), stirred 10 minutes then treated with methyl 4-bromomethylbenzoate (45 mg, 0.19 mmol) and stirred overnight. The mixture was diluted with water (1 ml), extracted twice with chloroform (1 ml) and the combined extracts washed with water and evaporated to give the title compound identical with that described in Example 28b (Method B).

c) O-3-Cyanobenzyl-1-(4-methoxycarbonylbenzyl)serotonin.

N-t-Butoxycarbonyl-O-3-cyanobenzyl-1-(4-methoxycarbonylbenzyl)-serotonin (76 mg) was dissolved in 4N hydrogen chloride in dioxan (1 ml) and set aside at RT for 1 h. The solution was evaporated to dryness to provide the title compound as a colourless crystalline solid (68 mg) MS (ES+) m/e 440 [M+H]⁺, 481.

d) 1-(4-Carboxylbenzyl)-O-3-cyanobenzylserotonin

2.5N Aqueous sodium hydroxide (1 ml) was added to O-3-Cyanobenzyl-1-(4-methoxycarbonyl-benzyl)serotonin (68 mg) in methanol (2 ml) and stirred for 2.5 h at RT. The reaction was quenched with hydrochloric acid, concentrated in vacuo, diluted with DMSO and the title compound isolated by preperative HPLC (gradient, 10-90% acetonitrile/water+0.1% trifluoroacetic acid) (29 mg, 48%) MS (ES+) m/e 426 [M+H]⁺, 467.

### EXAMPLE 29

### Preparation of 1-(4-methoxy)benzenesulfonyl-3-(2-aminoethyl)-5-(1-naphthyl) methyloxyindole trifluoroacetate

To a solution of 3-[2-(tert-butoxycarbonylamino)ethyl]-5-hydroxyindole (0.25 g, 0.9 mmol) in acetone (10 mL) was added 1-chloromethylnaphthalene (0.18 g, 1.0 mmol), potassium carbonate (0.38 g, 2.7 mmol), and potassium iodide (0.015 g, 0.09 mmol). The mixture was allowed to stir at room temperature for 5 d, then was filtered through Celite and concentrated. The residue was dissolved in methylene chloride (2 mL). Tetrabutyl-ammonium hydrogen sulfate (0.006 g, 0.017 mmol) and powdered sodium hydroxide (0.023 g, 0.60 mmol) were added, and the mixture was allowed to stir for 1 h. A 0.12M solution of 4-methoxybenzenesulfonyl chloride in methylene chloride (1 mL) was added, and the mixture stirred for 16 h. Water was added to the mixture and the organic layer extracted, dried, and concentrated. The residue was diluted with diethyl ether (5 mL) and treated with 4 N HCl in dioxane (2 mL). The solution stirred at room temperature for 2 h and was concentrated. The residue was purified by preparative HPLC (step gradient, 10-90% acetonitrile/water+0.1% trifluoroacetic acid, 15 min run) to furnish the title compound. MS (ES+) m/e 487 [M+H]⁺, 528.

### EXAMPLE 30

### Preparation of 1-(4-methoxy)benzenesulfonyl-3-(2-aminoethyl)-5-(2-naphthyl) methyloxyindole trifluoroacetate

Following the procedure of Example 29, except substituting 2-chloromethyl-naphthalene for 1-chloromethylnaphthalene, the title compound was prepared. MS (ES+) m/e 487 [M+H]⁺, 528.

### EXAMPLE 31

### Preparation of 1-(8-quinoline)sulfonyl-3-(2-aminoethyl)-5-(2-naphthyl)methyloxyindole trifluoroacetate

Following the procedure of Example 29, except substituting 2-chloromethyl-naphthalene for 1-chloromethylnaphthalene and 8-quinolinesulfonyl chloride for 4-methoxybenzenesulfonyl chloride, the title compound was prepared. MS (ES+) m/e 508 [M+H]⁺, 549.

### EXAMPLE 32

### Preparation of 1-(3-chloro-4-fluoro)benzenesulfonyl-3-(2-aminoethyl)-5-(2-naphthyl) methyloxyindole trifluoroacetate

Following the procedure of Example 29, except substituting 2-chloromethyl-naphthalene for 1-chloromethylnaphthalene and 3-chloro-4-fluorobenzenesulfonyl chloride for 4-methoxybenzenesulfonyl chloride, the title compound was prepared. MS (ES+) m/e 509 [M]⁺, 511 [M+2]⁺, 550.

### EXAMPLE 33

### Preparation of 1-(8-quinoline)sulfonvi-3-(2-aminoethyl)-5-(2-biphenyl)methyloxyindole trifluoroacetate

Following the procedure of Example 29, except substituting 2-biphenylmethyl bromide for 1-chloromethylnaphthalene and 8-quinolinesulfonyl chloride for 4-methoxybenzenesulfonyl chloride, the title compound was prepared. MS (ES+) m/e 534 [M+H]⁺, 575.

### EXAMPLE 34

### Preparation of 1-(2-chloro-4-fluoro)benzenesulfonyl-3-(2-aminoethyl)-5-(2-biphenyl)methyloxyindole trifluoroacetate

Following the procedure of Example 29, except substituting 2-biphenylmethyl bromide for 1-chloromethylnaphthalene and 2-chloro-4-fluorobenzenesulfonyl chloride for 4-methoxybenzenesulfonyl chloride, the title compound was prepared. MS (ES+) m/e 535 [M]⁺, 537 [M+2]⁺, 576.

### EXAMPLE 35

### Preparation of 1-(4-methoxy)benzenesulfonyl-3-(2-aminoethyl)-5-(4-biphenyl) methyloxyindole trifluoroacetate

Following the procedure of Example 29, except substituting 4-biphenylmethyl chloride for 1-chloromethylnaphthalene, the title compound was prepared. MS (ES+) m/e 513 [M+H]⁺, 554.

### EXAMPLE 36

### Preparation of 1-(2-thienyl)sulfonyl-3-(2-aminoethyl)-5-(4-biphenyl)methyloxyindole trifluoroacetate

Following the procedure of Example 29, except substituting 4-biphenylmethyl chloride for 1-chloromethylnaphthalene and 2-thiophenesulfonyl chloride for 4-methoxybenzenesulfonyl chloride, the title compound was prepared. MS (ES+) m/e 489 [M+H]⁺, 530.

### EXAMPLE 37

### Preparation of 1-(2-thienyl)sulfonyl-3-(2-aminoethyl)-5-[(4-tert-butyl)phenyl] methyloxyindole trifluoroacetate

Following the procedure of Example 29, except substituting 4-tert-butylbenzyl bromide for 1-chloromethylnaphthalene and 2-thiophenesulfonyl chloride for 4-methoxybenzenesulfonyl chloride, the title compound was prepared. MS (ES+) m/e 469 [M+H]⁺, 510.

### EXAMPLE 38

### Preparation of 3-(2-aminoethyl)-5-[(3-phenoxy)benzyloxylindole hydrochloride

To a solution of 3-[2-(tert-butoxycarbonylamino)ethyl]-5-hydroxyindole (0.14 g, 0.50 mmol), triphenylphosphine (0.20 g, 0.75 mmol), and 3-phenoxybenzyl alcohol (0.10 g, 0.50 mmol) in methylene chloride at 0° C. was added diisopropyl azodicarboxylate (0.11 mL, 0.55 mmol) dropwise. The reaction was allowed to warm to room temperature and stirred for 24 h. It was concentrated and purified by flash chromatography (silica gel, 30% ethyl acetate/hexane) to furnish the crude BOC-protected product, which was then dissolved in diethyl ether (5 mL). 4 N HCl in dioxane (1 mL) was added, and the solution was allowed to stir for 1 h. Additional 4 N HCl in dioxane (0.5 mL) was added, and the reaction stirred for 30 min. It was diluted with diethyl ether (5 mL) and the precipitated product (0.01 g, 5% yield) was filtered and washed with diethyl ether. MS (ES+) m/e 359 [M+H]⁺, 400

### EXAMPLE 39

### Preparation of 3-(2-aminoethyl)-5-[(2-naphthyl)methyloxy]indole hydrochloride

Following the procedure of Example 38, except substituting 2-naphthylmethyl alcohol for 3-phenoxybenzyl alcohol, the title compound was prepared as a gray solid. MS (ES+) m/e 317 [M+H]⁺, 633

### EXAMPLE 40

### Preparation of 3-(2-aminoethyl)-5-[(2-phenyl)benzyloxy]indole hydrochloride

Following the procedure of Example 38, except substituting 2-phenylbenzyl alcohol for 3-phenoxybenzyl alcohol, the title compound was prepared as a gray solid. MS (ES+) m/e 343 [M+H]⁺, 384

### EXAMPLE 41

### Preparation of 3-(2-aminoethyl)-5-[(4-phenyl)benzyloxy]indole hydrochloride

Following the procedure of Example 38, except substituting 4-phenylbenzyl alcohol for 3-phenoxybenzyl alcohol, the title compound was prepared as a gray solid. MS (ES+) m/e 343 [M+H]⁺, 384

### EXAMPLE 42

### Preparation of 1-(carbamoylmethyl)-3-(2-aminoethyl)-5-(2-naphthyl)indole trifluoroacetate

a) 1-(carbamoylmethyl)-3-(2-aminoethyl)-5-(2-naphthyl)indole trifluoroacetate

The compound of Example 2(a) (0.074 g, 0.19 mmol) was dissolved in anhydrous dimethylformamide (3 mL). Cesium carbonate (0.16 g, 0.48 mmol) was added, followed by 2-bromoacetamide (0.04 g, 0.29 mmol) in anhydrous dimethylformamide (0.5 mL). The mixture stirred at room temperature for 72 h. Additional 2-bromoacetamide (0.08 g, 0.58 mmol) and cesium carbonate (0.062 g, 0.19 mmol) were added, and stirring continued at 50° C. overnight. The mixture was cooled to room temperature, quenched with 1 N HCl (5 mL), and extracted with ethyl acetate (10 mL). The organic portion was washed with brine (10 mL), dried over Na₂SO₄, filtered, and concentrated. The crude residue was purified by flash chromatography (silica gel, step gradient, 67% ethyl acetate/hexane—100% ethyl acetate). The clean product was dissolved into cold 1:1 trifluoroacetic acid/methylene chloride (3 mL) and stirred at room temperature for 45 min. The solution was concentrated, diluted with diethyl ether, and concentrated again. The residue was taken up into 1,4-dioxane (1.5 mL) and slowly diluted with diethyl ether (20 mL) to give a white slurry. The slurry stirred for 1 h and was filtered. The solid was rinsed with diethyl ether and vacuum dried over potassium hydroxide to furnish the title compound (0.02 g, 23%) as a white solid. MS (ES+) m/e 344 [M+H]⁺, 687

### EXAMPLE 43

### Preparation of 1-(N-methylcarbamoylmethyl)-3-(2-aminoethyl)-5-(1-naphthyl)indole trifluoroacetate

1-(N-methylcarbamoylmethyl)-3-(2-aminoethyl)-5-(1-naphthyl)indole trifluoroacetate

Following the procedure of Example 42(a), except substituting the compound of Example 4(a) for the compound of Example 2(a) and 2-chloro-N-methylacetamide for 2-bromoacetamide, the title compound was prepared (0.018 g, 38%) as a white solid. MS (ES+) m/e 358 [M+H]⁺, 399

### EXAMPLE 44

### Preparation of 1-[(3-carbamoylphenyl)methyl]-3-(2-aminoethyl)-5-(1-naphthyl)indole hydrochloride

a) 1-[(3-cyanophenyl)methyl]-3-(2-tert-butoxycarbonylamino)ethyl]-5-(1-naphthyl)indole

The compound of Example 4(a) (0.11 g, 0.28 mmol) was dissolved in anhydrous dimethylformamide (5 mL). Cesium carbonate (0.46 g, 1.41 mmol) was added, followed by ?-bromo-m-tolunitrile (0.083 g, 0.42 mmol). The mixture was heated to 50° C. and allowed to stir at this temperature for 18 h. It was cooled to room temperature, diluted with water (10 mL) and extracted with ethyl acetate (10 mL). The organic portion was washed with water (10 mL) and brine (10 mL), dried over Na₂SO₄, filtered, and concentrated to a yellow oil. Purification by flash chromatography (silica gel, step gradient, 33%-50% ethyl acetate/hexane) furnished the product (0.11 g, 77%) as a colorless resin. ¹H NMR (CDCl₃) δ7.98-7.84 (3H, m), 7.73-7.72 (1H, m), 7.68-7.33 (10H, m), 7.04 (1H, s), 5.37 (2H, s), 4.64-4.61 (1H, br s), 3.49-3.46 (2H, m), 2.98 (2H, t, J=6.8 Hz), 1.36 (9H, s)

b) 1-[(3-carbamoylphenyl)methyl]-3-(2-tert-butoxycarbonylamino)ethyl]-5-(1-naphthyl)indole

A solution of the compound of Example 44(a) (0.096 g, 0.19 mmol) in 6N sodium hydroxide (7.6 mL, 45.6 mmol) and ethanol (30 mL) was cooled to 0° C. and treated with 30% hydrogen peroxide (0.077 mL, 0.67 mmol). The mixture stirred for 15 min at room temperature, then was heated to 70° C. Tetrabutylammonium hydrogen sulfate (??amount) was added to the warm solution, and it continued to stir at this temperature for an additional 1.5 h. The mixture was cooled slightly and neutralized with 10% H₂SO_{4.} The slurry was reduced to a lesser volume (20 mL), taken into water (20 mL) , and extracted with ethyl acetate (30 mL). The organic portion was washed with brine (25 mL), dried over Na₂SO₄, filtered, and concentrated. The yellow residue was purified by flash chromatography (silica gel, 100% ethyl acetate) to furnish the product (0.024 g, 24%) as a colorless oil. ¹H NMR (CDCl₃) δ7.99-7.70 (6H, m), 7.55-7.33 (10H, m), 7.06 (1H, s), 5.38 (2H, s), 4.67 (1H, br s), 3.50-3.31 (2H, m), 2.96 (2H, t, J=6.4 Hz), 1.29 (9H, s)

c) 1-[(3-carbamoylphenyl)methyl]-3-(2-aminoethyl)-5-(1-naphthyl) indole hydrochloride

The compound of Example 44(b) (0.03 g, 0.05 mmol) was taken up in diethyl ether (10 mL) and treated with 4 N HCl in dioxane (1 mL). The slurry stirred for 1 h and was filtered. The solid product was rinsed with diethyl ether and vacuum dried over potassium hydroxide to provide the title compound (0.018 g, 87%) as an off-white solid. ¹H NMR (CDCl₃) δ8.01-7.77 (8H, m), 7.69-7.68 (1H, m), 7.59-7.41 (10H, m), 7.24 (1H, dd, J=8.4 Hz, J=1.4 Hz), 5.48 (2H, s), 3.56-3.05 (4H, m).

### EXAMPLE 45

### Preparation of 1-[(3-carboxyphenyl)methyl]-3-(2-aminoethyl)-5-(1-naphthyl)indole hydrochloride

The second spot isolated from flash chromatography in Example 44(b) was identified as 1-[(3-carboxyphenyl)methyl]-3-[(2-tert-butoxycarbonylamino)ethyl]-5-(1-naphthyl) indole by standard characterization methods. Following the procedure of Example 44(c), the title compound was prepared as a cream-colored solid. MS (ES+) m/e 421 [M+H]⁺, 462

### EXAMPLE 46

### Preparation of 1-[(3-morpholinoylphenyl)methyl]-3-(2-aminoethyl)-5-(1-naphthyl)indole hydrochloride

The compound of Example 4(a) (0.15 g, 0.38 mmol) was dissolved in anhydrous dimethylformamide (1.5 mL). Cesium carbonate (0.62 g, 1.91 mmol) was added, followed by methyl 3-(bromomethyl)benzoate (0.13 g, 0.57 mmol). The mixture was allowed to stir at room temperature for 20 h. It was quenched with water (5 mL) and extracted into ethyl acetate (10 mL). The organic portion was washed with brine (10 mL), dried over Na₂SO₄, filtered, and concentrated. The pale yellow oil was purified by flash chromatography (silica gel, step gradient, 25%-33% ethyl acetate/hexane).

The clean product (0.17 g, 0.32 mmol) and sodium hydroxide (0.064 g, 1.61 mmol) were heated at reflux in 5:1 methanol/water (25 mL) for 10 h. The mixture was cooled to room temperature and concentrated. The residue was taken into 1 N HCl (30 mL) and extracted with ethyl acetate (30 mL). The organic portion was washed with brine (25 mL), dried over Na₂SO₄, filtered, and concentrated to a white foam.

This product (0.05 g, 0.10 mmol) was dissolved in anhydrous dimethylformamide (3 mL) and treated with N-methylmorpholine (0.012 mL, 0.11 mmol), HOBT (0.015 g, 0.11 mmol) and EDCI (0.028 g, 0.14 mmol). The mixture stirred for 10 min and was treated with morpholine (0.013 mL, 0.14 mmol). The resulting mixture was allowed to stir at room temperature for 18 h. It was quenched with 1 N HCl (5 mL) and extracted into ethyl acetate (10 mL). The organic portion was washed with 1 N HCl (5 mL) and brine (5 mL), dried over Na₂SO₄, filtered, and concentrated. The crude oil was purified by flash chromatography (silica gel, step gradient, 50% ethyl acetate/hexane—100% ethyl acetate) to give the product (0.032 g, 0.05 mmol), which was dissolved in 4 N HCl in dioxane (1.5 mL) and stirred for 1 h. The mixture was diluted with diethyl ether and the solids collected by filtration and vacuum dried to furnish the title compound (0.02 g, 32%) as a white solid. MS (ES+) m/e 490 [M+H]⁺, 979

### EXAMPLE 47

### Preparation of 1-[(3-(3-trifluoromethylbenzyl)carbamoylphenyl)methyl]-3-(2-aminoethyl)-5-(1-naphthyl)indole hydrochloride

Following the procedure of Example 46, except substituting 3-(trifluoromethyl) benzylamine for morpholine, the title compound was prepared as a white solid. MS (ES+) m/e 578 [M+H]⁺, 600

### EXAMPLE 48

### Preparation of 1-[(3-carbamoylphenyl)methyl]-3-(2-aminoethyl)-5-(4-dibenzofuranyl)indole hydrochloride

a) 1-[(3-carbamoylphenyl)methyl]-3-(2-aminoethyl)-5-(4-dibenzofuranyl)indole hydrochloride

The compound of Example 11(a) (0.075 g, 0.18 mmol) was dissolved in anhydrous dimethylformamide (3 mL). Cesium carbonate (0.29 g, 0.88 mmol) was added, followed by 3-chloromethylbenzamide (0.036 g, 0.21 mmol), and the mixture was allowed to stir for 2 d. It was diluted with water (10 mL) and extracted into ethyl acetate (10 mL). The organic portion was washed with brine (10 mL), dried over Na₂SO₄, filtered, and concentrated to a crude oil which was purified by flash chromatography (silica gel, 50% ethyl acetate/hexane). The clean product was dissolved in 4 N HCl in dioxane (1 mL) and allowed to stir for 1 h. The mixture was diluted with diethyl ether and the solids collected by filtration. The solid was rinsed with additional diethyl ether and vacuum dried over potassium hydroxide to furnish the product (0.012 g, 14%) as an off-white powder. MS (ES+) m/e 460 [M+H]⁺, 919.

### EXAMPLE 49

### Preparation of 1-[(4-carbamoylphenyl)methyl]-3-(2-aminoethyl)-5-(4-dibenzofuranyl)indole hydrochloride

Following the procedure of Example 48, except substituting 4-chloromethyl-benzamide for 3-chloromethylbenzamide, the title compound was prepared (0.040 g, 44%) as a white powder. MS (ES+) m/e 460 [M+H]⁺, 482

### EXAMPLE 50

### Preparation of 1-(carbamoylmethyl)-3-(2-aminoethyl)-5-(4-dibenzofuranyl)indole hydrochloride

Following the procedure of Example 48, except substituting 2-bromoacetamide for 3-chloromethylbenzamide, the title compound was prepared (0.017 g, 30%) as a white solid. MS (ES+) m/e 384 [M+H]⁺, 767

### EXAMPLE 51

### Preparation of 1-(4-carbomethoxyphenyl)methyl-3-(2-aminoethyl)-5-(4-dibenzofuranyl) indole hydrochloride

Following the procedure of Example 48, except substituting methyl 4-(bromomethyl) benzoate for 3-chloromethylbenzamide, the title compound was prepared as an off-white solid. MS (ES+) m/e 475 [M+H]⁺, 516

### EXAMPLE 52

### Preparation of 1-(3-carbomethoxyphenyl)methyl-3-(2-aminoethyl)-5-(4-dibenzofuranyl) indole hydrochloride

Following the procedure of Example 48, except substituting methyl-3-(bromomethyl) benzoate for 3-chloromethylbenzamide, the title compound was prepared as an off-white solid. MS (ES+) m/e 475 [M+H]⁺, 497

### EXAMPLE 53

### Preparation of 1-(2-cyanophenyl)methyl-3-(2-aminoethyl)-5-(4-dibenzofuranyl)indole hydrochloride

Following the procedure of Example 48, except substituting δ-bromo-o-tolunitrile for 3-chloromethylbenzamide, the title compound was prepared (0.011 g, 84%) as a cream-colored solid. MS (ES) m/e 442 [M+H]⁺, 883

### EXAMPLE 54

### Preparation of 1-(3-cyanophenyl)methyl-3-(2-aminoethyl)-5-(4-dibenzofuranyl)indole hydrochloride

Following the procedure of Example 48, except substituting δ-bromo-m-tolunitrile for 3-chloromethylbenzamide, the title compound was prepared (0.019 g, 34%) as a light gray solid. MS (ES+) m/e 442 [M+H]⁺, 883

### EXAMPLE 55

### Preparation of 1-(4-acetamidophenyl)methyl-3-(2-aminoethyl)-5-(4-dibenzofuranyl)indole hydrochloride

Following the procedure of Example 48, except substituting 4-acetamidobenzyl chloride for 3-chloromethylbenzamide, the title compound was prepared (0.005 g, 11%) as a cream-colored solid. MS (ES+) m/e 474 [M+H]⁺, 515

### EXAMPLE 56

### Preparation of 1-(5-cyanopentyl)-3-(2-aminoethyl)-5-(4-dibenzofuranyl)indole hydrochloride

a) 1-(5-cyanopentyl)-3-(2-tert-butoxycarbonylamino)-5-(4-dibenzofuranyl)indole

The compound of Example 1 1(a) (0.10 g, 0.24 mmol) and 6-bromohexanenitrile (0.064 mL, 0.48 mmol) were dissolved in anhydrous tetrahydrofuran (6 mL) and treated with 0.5M potassium hexamethyldisilazide in toluene(1.00 mL, 0.48 mmol). The mixture was allowed to stir at room temperature for 2 d. An additional aliquot of 6-bromohexane-nitrile (0.032 mL, 0.24 mmol) was added, and the mixture was heated to reflux where it was allowed to stir for 3 h. It was cooled to room temperature, quenched with water (5 mL) and extracted with ethyl acetate (10 mL). The organic portion was washed with brine (5 mL), dried over Na₂SO₄, filtered, and concentrated. The crude residue was purified by flash chromatography (silica gel, step gradient, 25-33% ethyl acetate/hexane) to furnish the product (0.043 g, 34%) as a colorless oil. ¹H NMR (CDCl₃) 8 8.07 (1H, d, J=1.2 Hz), 8.02-7.99 (1H, m), 7.92 (1H, dd, J=7.6 Hz, J=1.3 Hz), 7.79 (1H, dd, J=8.5 Hz, J=1.5 Hz), 7.67-7.59 (2H, m), 7.49-7.34 (4H, m), 6.99 (1H, s), 4.70-4.60 (1H, m), 4.16 (2H, t, J=6.9 Hz), 3.52-3.50 (2H, m), 3.02 (2H, t, J=6.6 Hz), 2.34 (2H, t, J=7.0 Hz), 1.94-1.87 (2H, m), 1.74-1.64 (2H, m), 1.56-1.48 (2H, m), 1.39 (9H, s)

b) 1-(5-cyanopentyl)-3-(2-aminoethyl)-5-(4-dibenzofuranyl)indole hydrochloride

Following the procedure of Example 44(c), the title compound was prepared (0.014, 37%) as a tan solid. MS (ES) m/e 422 [M+H]⁺, 463

### EXAMPLE 57

### Preparation of 1-(4-cyanobutyl)-3-(2-aminoethyl)-5-(4-dibenzofuranyl)indole hydrochloride

Following the procedure of Example 56(a)-56(b), except substituting 5-bromovaleronitrile for 6-bromohexanenitrile, the title compound was prepared (0.022 g, 40%) as a cream-colored solid. MS (ES+) m/e 408 [M+H]⁺, 815

### EXAMPLE 58

### Preparation of 1-(2-carbamoylethyl)-3-(2-aminoethyl)-5-(4-dibenzofuranyl)indole hydrochloride

Following the procedure of Example 56(a)-56(b), except substituting 3-iodopropionamide for 6-bromohexanenitrile, the title compound was prepared as a tan solid. MS (ES+) m/e 398 [M+H]⁺, 795

### EXAMPLE 59

### Preparation of 1-[(2-carbamoylphenyl)methyl]-3-(2-aminoethyl)-5-(4-dibenzofuranyl)indole hydrochloride

Following the procedure of Example 44(a)-44(c), except substituting the compound of Example 10 for the compound of Example 4 and α-bromo-o-tolunitrile for α-bromo-m-tolunitrile, the title compound was prepared as a white powder. MS (ES+) m/e 460 [M+H]⁺, 482

### EXAMPLE 60

### Preparation of 1-ethyl-3-(2-aminoethyl)-5-(4-dibenzofuranyl)indole hydrochloride

a) 1-ethyl-3-[(2-tert-butoxycarbonylamino)ethyl]-5-(4-dibenzofuranyl)indole

1-Phenylsulfonyl-3-[2-(tert-butoxycarbonylamino)ethyl]-5-bromoindole (intermediate in Example 10(a)) (3.00 g, 6.26 mmol) and cesium carbonate (8.16 g, 25.0 mmol) were dissolved in 10:1 ethylene glycol dimethyl ether:water (110 mL) and the solution was degassed with argon. Pd(PPh₃)₄ was added (0.22 g, 0.19 mmol), followed by dibenzofuran-4-boronic acid (2.65 g, 12.5 mmol). The mixture was again degassed with argon over 10 min and then heated at reflux for 20 h. It was cooled, filtered, and partitioned between brine (50 mL) and diethyl ether (50 mL). The organic portion was separated and washed with saturated sodium bicarbonate (50 mL) and brine (50 mL), dried over Na₂SO₄, filtered, and concentrated to a crude yellow oil. Purification by flash chromatography (silica gel, step gradient, 25-33% ethyl acetate/hexane) furnished the title compound as the minor product (0.11 g, 5%). MS (ES+) m/e 455 [M+H]⁺, 477

b) 1-ethyl-3-(2-aminoethyl)-5-(4-dibenzofuranyl)indole hydrochloride

Following the procedure of Example 44(c), the title compound was prepared (0.04 g, 67%) as a white solid. MS (ES+) m/e 355 [M+H]⁺, 396

### EXAMPLE 61

### Preparation of 3-(2-aminoethyl)-5-(4-dibenzofuranyl)indole-1-pentanoic acid hydrochloride

The compound of Example 57(a) (0.10 g, 0.20 mmol) was dissolved in methanol (5.5 mL). 25% aqueous sodium hydroxide (1.5 mL) was added, and the mixture was heated at 100° C. for 18 h. It was cooled to 0° C. and diluted with 1 N HCl until pH=1. The mixture was extracted with ethyl acetate (20 mL), and the organic portion was washed with brine (20 mL), dried over Na₂SO₄, filtered, and concentrated. The crude product was dissolved in 4 N HCl in dioxane (1 mL) and allowed to stir at room temperature for 1 h. The mixture was diluted with diethyl ether and allowed to stir for an additional 30 min. The solids were filtered and washed with diethyl ether, then vacuum dried over potassium hydroxide to furnish the title compound (0.045 g, 48%) as a white solid. MS (ES+) m/e 427 [M+H]⁺, 468

### EXAMPLE 62

### Preparation of 1-[(3-carboxyphenyl)methyl]-3-(2-aminoethyl)-5-(benzothiophen-2-yl)indole hydrochloride

The compound of Example 17(a) (0.08 g, 0.20 mmol) was dissolved in anhydrous dimethylformamide (2 mL) and treated with cesium carbonate (0.33 g, 1.02 mmol). The mixture stirred for 15 min, at which time methyl 3-(bromomethyl)benzoate(0.07 g, 0.31 mmol) was added. The mixture was allowed to stir at room temperature overnight. It was quenched with water (5 mL) and extracted with ethyl acetate (5 mL). The organic portion was washed with brine (5 mL), dried over Na₂SO₄, filtered, and concentrated. Purification by flash chromatography (silica gel, step gradient, 25-33% ethyl acetate/hexane) furnished the N-alkylated product, which was then dissolved in methanol (10 mL) and 1 N sodium hydroxide (10 mL). The mixture was heated at reflux for 18 h, then cooled and concentrated. The residue was taken up in 3 N HCl (20 mL) and extracted with ethyl acetate (20 mL). The organic portion was washed with brine (20 mL), dried over Na₂SO₄, filtered, and concentrated. The crude product was dissolved in 4 N HCl in dioxane (1 mL) and allowed to stir at room temperature for 1 h. The white slurry was diluted with diethyl ether and the solids collected by filtration, washed with diethyl ether, and vacuum dried over potassium hydroxide to furnish the title compound (0.05 g, 54%) as a white powder. MS (ES+) m/e 427 [M+H]⁺, 468

### EXAMPLE 63

### Preparation of 1-[(4-carboxyphenyl)methyl]-3-(2-aminoethyl)-5-(benzothiophen-2-yl)indole hydrochloride

Following the procedure of Example 62, except substituting methyl 4-(bromomethyl)benzoate for substituting methyl 3-(bromomethyl)benzoate, the title compound was prepared as a white powder. MS (ES+) m/e 427 [M+H]⁺, 468

### EXAMPLE 64

### Preparation of 1-[(3-carboxyphenylemethyl]-3-(2-aminoethyl)-5-(4-dibenzofuranyl)indole hydrochloride

Following the procedure of Example 62, except substituting the compound of Example 11(a) for the compound of Example 17(a), the title compound was prepared as a white powder. MS (ES+) m/e 461 [M+H]⁺, 502

### EXAMPLE 65

### Preparation of 3-(2-aminoethyl)-5-(4-dibenzofuranyl)indole-1-butanoic acid hydrochloride

a) ethyl 3-(2-tert-butoxycarbonylamino)-5-(4-dibenzofuranyl)indole-1-butanoate

A solution of the compound of Example 11(a) (0.078 g, 0.18 mmol) in anhydrous dimethylformarnide (1.5 mL) was treated with sodium hydride (0.007 g, 0.18 mmol, 60% in mineral oil). The mixture stirred for 5 min, and ethyl 4-bromobutyrate (0.053 mL, 0.37 mmol) was added. The reaction was allowed to stir at room temperature for 24 h. Additional ethyl 4-bromobutyrate (0.10 mL, 0.74 mmol) was added, and stirring continued for another 24 h. The mixture was partitioned between water (5 mL) and ethyl acetate (5 mL). The organic portion was separated and washed with brine (5 mL), dried over Na₂SO₄, filtered, and concentrated. Purification by preparative HPLC (step gradient, 10-90% acetonitrile/water+0.1% trifluoroacetic acid, 15 min run) furnished the product (0.018 g, 18%). MS (ES+) m/e 541 [M+H]⁺, 563

b) 3-(2-tert-butoxycarbonylamino)-5-(4-dibenzofuranyl)indole-1-butanoic acid

The compound of Example 65(a) was taken into 1:1 1 N sodium hydroxide: methanol (3 mL) and heated at reflux for 10 min. The resulting clear solution was allowed to cool to room temperature and the solvent reduced in vacuo to approximately half the volume. This residue was treated with 7 drops of concentrated HCl and stirred for 20 min. The mixture was diluted with water and centrifuged. The precipitate was filtered and vacuum dried to furnish the product (0.014 g, 100%) as an off-white solid. MS (ES+) m/e 512 [M+H]⁺, 535

c) 3-(2-aminoethyl)-5-(4-dibenzofuranyl)indole-1-butanoic acid hydrochloride

Following the procedure of Example 44(c), the title compound was prepared (0.004 g, 32%) as an off-white solid. MS (ES+) m/e 413 [M+H]⁺, 454

### EXAMPLE 66

### Preparation of 1-[(N-(3-trifluoromethylphenyl)methyl)acetamido]-3-(2-aminoethyl)-5-(1-naphthyl)indole

The compound of Example 4(a) (1.28 g, 3.31 mmol) was dissolved in anhydrous dimethylformamide (10 mL). Cesium carbonate (2.70 g, 8.28 mmol) was added, followed by ethyl 2-bromoacetate (0.55 mL, 4.97 mmol). The mixture was allowed to stir at room temperature for 20 h. It was quenched with water (10 mL) and extracted with ethyl acetate (20 mL). The organic portion was separated and washed with brine (20 mL), dried over Na₂SO₄, filtered, and concentrated. Purification by flash chromatography (silica gel, step gradient, 25-33% ethyl acetate/hexane) furnished the N-alkylated product, which was dissolved in 5:1 methanol:1 N sodium hydroxide (50 mL) and heated to reflux. After 14 h, the mixture was cooled to room temperature and concentrated. The residue was taken into 1 N HCl (20 mL) and extracted with ethyl acetate (20 mL). The organic portion was washed with 1 N HCl (10 mL) and brine (10 mL), dried over Na₂SO₄, filtered, and concentrated.

The crude acid (0.04 g, 0.09 mmol) was dissolved in dimethylformamide (0.5 mL) and treated with 3-(trifluoromethyl)benzylamine (0.016 g, 0.09 mmol), N-methylmorpholine (0.01 g, 0.10 mmnol), HOBT (0.014 g, 0.10 mmol), and EDCI (0.026 g, 0.13 mmol). The mixture was allowed to stir at room temperature for 3 d. It was diluted with 1:1 2 N HCl:brine (1 mL) and extracted with ethyl acetate (3×1 mL). The organic portions were concentrated, and the crude residue was purified by preparative HPLC (step gradient, 10-90% acetonitrile/water+0.1% trifluoroaetic acid, 15 min run) to furnish the title compound dissolved in 4 N HCl in dioxane (1 mL) and allowed to stir at room temperature for 1 h. The white slurry was diluted with diethyl ether and the solids collected by filtration, washed with diethyl ether, and vacuum dried over potassium hydroxide to furnish the title compound as a white powder. MS (ES+) m/e 502 [M+H]⁺, 1002

### EXAMPLE 67

### Preparation of 1-[(N-(4-pyridyl)methyl)acetamido]-3-(2-aminoethyl)-5-(1-naphthyl)indole

Following the procedure of Example 66, except substituting 4-aminomethylpyridine for 3-(trifluoromethyl)benzylamine, the title compound was prepared. MS (ES+) m/e 435 [M+H]⁺, 868

### EXAMPLE 68

### Preparation of 1-[(N-(3-methoxyphenyl)methyl)acetamido]-3-(2-aminoethyl)-5-(1-naphthyl)indole

Following the procedure of Example 66, except substituting 3-methoxybenzylamine for 3-(trifluoromethyl)benzylamine, the title compound was prepared.

### EXAMPLE 69

### Preparation of 1-[(N-(4-sulfonamidophenyl)methyl)acetamido]-3-(2-aminoethyl)-5-(1-naphthyl)indole

Following the procedure of Example 66, except substituting 4-aminomethylbenzene-sulfonamide for 3-(trifluoromethyl)benzylamine, the title compound was prepared. MS (ES+) m/e 512 [M+H]⁺, 534

### EXAMPLE 70

### Preparation of 1-[(N-benzyl)acetamido]-3-(2-aminoethyl)-5-(1-naphthyl)indole

Following the procedure of Example 66, except substituting benzylamine for 3-(trifluoromethyl)benzylamine, the title compound was prepared.

### EXAMPLE 71

### Preparation of 1-[(N-(2,5-difluorophenyl)methyl)acetamido]-3-(2-aminoethyl)-5-(1-naphthyl)indole

Following the procedure of Example 66, except substituting 2,5-difluorobenzylamine for 3-(trifluoromethyl)benzylamine, the title compound was prepared. MS (ES+) m/e 470 [M+H]⁺, 511

### EXAMPLE 72

### Preparation of 1-[(N-(2,4-dichlorophenyl)methyl)acetamido]-3-(2-aminoethyl)-5-(1-naphthyl)indole

Following the procedure of Example 66, except substituting 2,4-dichlorobenzyl-amine for 3-(trifluoromethyl)benzylamine, the title compound was prepared. MS (ES+) m/e 502 [M]⁺, 504 [M+2]⁺, 524

### EXAMPLE 73

### Preparation of 1-[(N-(2-benzimidazole)methyl)acetamido]-3-(2-aminoethyl)-5-(1-naphthyl)indole

Following the procedure of Example 66, except substituting 2-aminomethyl-benzimidazole for 3-(trifluoromethyl)benzylamine, the title compound was prepared. MS (ES+) m/e 474 [M+H]⁺

### EXAMPLE 74

### Preparation of 1-[N-(3-pyridyl)acetamido]-3-(2-aminoethyl)-5-(1-naphthyl)indole

Following the procedure of Example 66, except substituting 3-aminopyridine for 3-(trifluoromethyl)benzylamine, the title compound was prepared. MS (ES+) m/e 421 [M+H]⁺, 462

### EXAMPLE 75

### Preparation of 1-[N-(2-thiazole)acetamido]-3-(2-aminoethyl)-5-(1-naphthyl)indole

Following the procedure of Example 66, except substituting 2-aminothiazole for 3-(trifluoromethyl)benzylamine, the title compound was prepared. MS (ES+) m/e 427 [M+H]⁺, 468

### EXAMPLE 76

### Preparation of 1-[(N-(2-thiophene)methyl)acetamido]-3-(2-aminoethyl)-5-(1-naphthyl)indole

Following the procedure of Example 66, except substituting 2-aminomethyl-thiophene for 3-(trifluoromethyl)benzylamine, the title compound was prepared. MS (ES+) m/e 440 [M+H]⁺, 481

### EXAMPLE 77

### Preparation of 1-[trans-(N-cyclopropylphenyl)acetamido]-3-(2-aminoethyl)-5-(1-naphthyl)indole

Following the procedure of Example 66, except substituting trans-1-amino-2-phenylcyclopropane for 3-(trifluoromethyl)benzylamine, the title compound was prepared. MS (ES+) m/e 460 [M+H]⁺, 482

### EXAMPLE 78

### Preparation of 1-[(N-(4-carboethoxy)piperidine)acetamido]-3-(2-aminoethyl)-5-(1-yl)indole

Following the procedure of Example 66, except substituting 4-ethoxycarbonyl-piperidine for 3-(trifluoromethyl)benzylamine, the title compound was prepared. MS (ES+) m/e 484 [M+H]⁺, 506

### EXAMPLE 79

### Preparation of 1-[N-(3-methoxyphenyl)acetamido]-3-(2-aminoethyl)-5-(4-dibenzofuranyl)indole

Following the procedure of Example 66, except substituting 3-methoxybenzylamine for 3-(trifluoromethyl)benzylamine and the compound of Example 1 1(a) for the compound of Example 4(a), the title compound was prepared. MS (ES+) m/e 490 [M+H]⁺, 531

| | | For Prep | |
|---|---|---|---|
| Example | Compound Name | see Ex.# | MS (ES+) m/e |
| 80 | 2-[3-(2-Aminoethyl)-5-naphtl-1-ylindol- | 66 | 484 (M + H) |
| | 1-yl]-N-naphth-1-ylmethylacetamide | | 967 (2M + H) |
| 81 | {2-[3-(2-Amino-ethyl)-5-dibenzofuran- | 79 | 498 (M + H) |
| | 4-yl-indol-1-yl]-ethanoylamino}-acetic | | 995 (2M + H) |
| | acid-tert-butylester | | |
| 82 | 2-[1-Benzenesulfonyl-5-(4-methyl- | 1 | 441 (M + H) |
| | naphthalen-1-yl)-1-H-indol-3-yl]- | | 482 (M + CH3CN + H) |
| | ethylamine | | |
| 83 | 4-[3-(2-Amino-ethyl)-1- | 1 | 442 (M + H) |
| | benzenesulfonyl-1-H-indol-5-yl]- | | 483 (M + CH3CN + H) |
| | naphthalen-1-ylamine | | |
| 84 | 2-[1-Benzenesulfonyl-5-(2- | 1 | 471 (M + H) |
| | methoxymethyl-naphthalen-1-yl)-1-H- | | 941 (2M + H) |
| | indol-3-yl]-ethylamine | | |
| 85 | 6-[3-(2-Amino-ethyl)-1- | 1 | 471 (M + H) |
| | benzenesulfonyl-1-H-indol-5-yl]- | | 941 (2M + H) |
| | naphthalen-2-ol | | |
| 86 | ′3-(2-Aminoethyl)-1-[N-(2- | 79 | 437 (M + H) |
| | cyanoethyl)acetamido]-5-(dibenzofuran- | | 873 (2M + H) |
| | 4-yl)-indole | | |
| 87 | ′3-(2-Aminoethyl)-1-[N-(2- | 79 | 495 (M + H) |
| | carbamoylethyl)-N- | | 983 (2M + H) |
| | cyclopropylacetamido]-5- | | |
| | (dibenzofuran-4-yl)indole | | |
| 88 | ′3-(2-Aminoethyl)-1-[N-(2-cyanoethyl)- | 79 | 477 (M + H) |
| | N-cyclopropylacetamido]-5- | | 953 (2M + H) |
| | (dibenzofuran-4-yl)indole | | |
| 89 | ′3-(2-Aminoethyl)-1-[N-(2- | 79 | 464 (M + H) |
| | furylmethyl)acetamido]-5- | | 927 (2M + H) |
| | (dibenzofuran-4-yl)indole | | |
| 90 | 2-[3-(2-Amino-ethyl)-5-dibenzofuran-4- | 79 | 495 (M + H) |
| | yl-indol-1-yl]-N-[2-(1-methyl- | | 989(2M + H) |
| | pyrrolidin-2-yl)-ethyl]-acetamide | | |
| 91 | 2-[3-(2-Amino-ethyl)-5-dibenzofuran-4- | 79 | 497 (M + H) |
| | yl-indol-1-yl]-N-(2-morpholin-4-yl- | | 993 (2M + H) |
| | ethyl)-acetamide | | |
| 92 | 2-[3-(2-Amino-ethyl)-5-dibenzofuran-4- | 79 | 430 (M + H) |
| | yl-indol-1-yl]-N-(2-fluoro-ethyl)- | | 859 (2M + H) |
| | acetamide | | |
| 93 | 2-[3-(2-Amino-ethyl)-5-dibenzofuran-4- | 79 | 442 (M + H) |
| | yl-indol-1-yl]-N-(2-methoxy-ethyl)- | | 883 (2M + H) |
| | acetamide | | |
| 94 | 2-[3-(2-Amino-ethyl)-5-dibenzofuran-4- | 79 | 422 (M + H) |
| | yl-indol-1-yl]-N-prop-2-ynyl-acetamide | | 843 (2M + H) |
| 95 | 2-[3-(2-Amino-ethyl)-5-dibenzofuran-4- | 79 | 456 (M + H) |
| | yl-indol-1-yl]-N-(4-hydroxy-butyl)- | | 911 (2M + H) |
| | acetamide | | |
| 96 | 2-[3-(2-Amino-ethyl)-5-dibenzofuran-4- | 79 | 492 (M + H) |
| | yl-indol-1-yl]-N-(3-imidazol-1-yl- | | 983 (2M + H) |
| | propyl)-acetamide | | |
| 97 | 2-[3-(2-Amino-ethyl)-5-dibenzofuran-4- | 79 | 466 (M + H) |
| | yl-indol-1-yl]-N-(2,2,2-trifluoro-ethyl)- | | 931 (2M + H) |
| | acetamide | | |
| 98 | N-(2-Amino-ethyl)-2-[3-(2-amino- | 79 | 427 (M + H) |
| | ethyl)-5-dibenzofuran-4-yl-indol-1-yl]- | | 853 (2M + H) |
| | acetamide | | |
| 99 | 2-[3-(2-Amino-ethyl)-5-dibenzofuran-4- | 79 | 451 (M + H) |
| | yl-indol-1-yl]-N -[1,2,4]triazol-4-yl- | | 901 (2M + H) |
| | acetamide | | |
| 100 | 2-[3-(2-Amino-ethyl)-5-dibenzofuran-4- | 79 | 509 (M + H) |
| | yl-indol-1-yl]-N-[3-(2-oxo-pyrrolidin-1- | | 1017 (2M + H) |
| | yl)-propyl]-acetamide | | |
| 101 | 2-[3-(2-Amino-ethyl)-5-dibenzofuran-4- | 79 | 478 (M + H) |
| | yl-indol-1-yl]-N-[2-(1-H-imidazol-4-yl)- | | 955 (2M + H) |
| | ethyl]-acetamide | | |
| 102 | 2-[3-(2-Amino-ethyl)-5-dibenzofuran-4- | 79 | 510 (M + H) |
| | yl-indol-1-yl]-N-(1,2-diethyl- | | 1019 (2M + H) |
| | pyrazolidin-4-yl)-acetamide | | |
| 103 | 2-[3-(2-Amino-ethyl)-5-dibenzofuran-4- | 79 | 494 (M + H) |
| | yl-indol-1-yl]-N-(2-thiophen-2-yl- | | 987 (2M + H) |
| | ethyl)-acetamide | | |
| 104 | 3-{2-[3-(2-Amino-ethyl)-5- | 79 | 456 (M + H) |
| | dibenzofuran-4-yl-indol-1-yl]- | | 911 (2M + H) |
| | ethanoylamino}-propionic acid | | |
| 105 | 2-[3-(2-Amino-ethyl)-5-dibenzofuran-4- | 79 | 455 (M + H) |
| | yl-indol-1-yl]-N-carbamoylmethyl-N- | | 909 (2M + H) |
| | methyl-acetamide | | |
| 106 | (S)-2-{2-[3-(2-Amino-ethyl)-5- | 79 | 484 (M + H) |
| | dibenzofuran-4-yl-indol-1-yl]- | | 967 (2M + H) |
| | ethanoylamino}-3-methyl-butyric acid | | |
| 107 | (S)-2-{2-[3-(2-Amino-ethyl)-5- | 79 | 456 (M + H) |
| | dibenzofuran-4-yl-indol-1-yl]- | | 911 (2M + H) |
| | ethanoylamino}-propionic acid | | |
| 108 | (S)-2-{2-[3-(2-Amino-ethyl)-5- | 79 | 498 (M + H) |
| | dibenzofuran-4-yl-indol-1-yl]- | | 995 (2M + H) |
| | ethanoylamino}-4-methyl-pentanoic | | |
| | acid | | |
| 109 | (2S,3S)-2-{2-[3-(2-Amino-ethyl)-5- | 79 | 498 (M + H) |
| | dibenzofuran-4-yl-indol-1-yl]- | | 995 (2M + H) |
| | ethanoylamino}-3-methyl-pentanoic | | |
| | acid | | |
| 110 | {2-[3-(2-Amino-ethyl)-5-dibenzofuran- | 79 | 442 (M + H) |
| | 4-yl-indol-1-yl]-ethanoylamino}-acetic | | 883 (2M + H) |
| | acid | | |
| 111 | 2-{2-[3-(2-Amino-ethyl)-5- | 79 | 470 (M + H) |
| | dibenzofuran-4-yl-indol-1-yl]- | | 939 (2M + H) |
| | ethanoylamino}-butyric acid | | |
| 112 | ({2-[3-(2-Amino-ethyl)-5-dibenzofuran- | 79 | 470 (M + H) |
| | 4-yl-indol-1-yl]-ethanoyl}-methyl- | | 939 (2M + H) |
| | amino)-acetic acid methyl ester | | |
| 113 | 2-[3-(2-Amino-ethyl)-5-dibenzofuran-4- | 79 | 441 (M + H) |
| | yl-indol-1-yl]-N-carbamoylmethyl- | | 881 (2M + H) |
| | acetamide | | |
| 114 | (S)-2-{2-[3-(2-Amino-ethyl)-5- | 79 | 455 (M + H) |
| | dibenzofuran-4-yl-indol-1-yl]- | | 909 (2M + H) |
| | ethanoylamino}-propionamide | | |
| 115 | 2-[3-(2-Amino-ethyl)-5-dibenzofuran-4- | 79 | 438 (M + H) |
| | yl-indol-1-yl]-N-cyclobutyl-acetamide | | 875 (2M + H) |
| 116 | 2-[3-(2-Amino-ethyl)-5-dibenzofuran-4- | 79 | 481 (M + H) |
| | yl-indol-1-yl]-N-(2-pyrrolidin-1-yl- | | 961 (2M + H) |
| | ethyl)-acetamide | | |
| 117 | (S)-2-{2-[3-(2-Amino-ethyl)-5- | 79 | 536 (M + H) |
| | dibenzofuran-4-yl-indol-1-yl]- | | 1071 (2M + H) |
| | ethanoylamino}-3-(1-H-imidazol-4-yl)- | | |
| | propionic acid methyl ester | | |
| 118 | 3-{2-[3-(2-Amino-ethyl)-5- | 79 | 455 (M + H) |
| | dibenzofuran-4-yl-indol-1-yl]- | | 909 (2M + H) |
| | ethanoylamino}-propionamide | | |
| 119 | 1-{2-[3-(2-Amino-ethyl)-5- | 79 | 524 (M + H) |
| | dibenzofuran-4-yl-indol-1-yl]- | | 1047 (2M + H) |
| | ethanoyl}-piperidine-3-carboxylic acid | | |
| | ethyl ester | | |
| 120 | 2-[3-(2-Amino-ethyl)-5-dibenzofuran-4- | 79 | 509 (M + H) |
| | yl-indol-1-yl]-1-(2- | | 1017 (2M + H) |
| | dimethylaminomethyl-piperidin-1-yl)- | | |
| | ethanone | | |
| 121 | 2-[3-(2-Amino-ethyl)-5-dibenzofuran-4- | 79 | 518 (M + H) |
| | yl-indol-1-yl]-N-((S)-1-hydroxymethyl- | | 1035 (2M + H) |
| | 2-phenyl-ethyl)-acetamide | | |
| 122 | 2-[3-(2-Amino-ethyl)-5-dibenzofuran-4- | 79 | 454 (M + H) |
| | yl-indol-1-yl]-1-morpholin-4-yl- | | 907 (2M + H) |
| | ethanone | | |
| 123 | 2-[3-(2-Amino-ethyl)-5-dibenzofuran-4- | 79 | 480 (M + H) |
| | yl-indol-1-yl]-N-cyclohexyl-N-methyl- | | 959 (2M + H) |
| | acetamide | | |
| 124 | 2-[3-(2-Amino-ethyl)-5-dibenzofuran-4- | 79 | 453 (M + H) |
| | yl-indol-1-yl]-N-pyrrolidin-1-yl- | | 905 (2M + H) |
| | acetamide | | |
| 125 | 2-[3-(2-Amino-ethyl)-5-dibenzofuran-4- | 79 | 497 (M + H) |
| | yl-indol-1-yl]-N-(4-guanidino-butyl)- | | 993 (2M + H) |
| | acetamide | | |
| 126 | 2-[5-(4-Methyl-naphthalen-1-yl)-1 H- | 2 | 301(M + H) |
| | indol-3-yl]-ethylamine | | 342 (M + CH3CN + H) |
| 127 | 6-[3-(2-Amino-ethyl)-1 H-indol-5-yl]- | 2 | 303 (M + H) |
| | naphthalen-2-ol | | 344 (M + CH3CN + H) |
| 128 | 7-[3-(2-Amino-ethyl)-1 H-indol-5-yl]-3- | 2 | 452 (M + H) |
| | hydroxy-naphthalene-2-carboxylic acid | | 493 (M + CH3CN + H) |
| | (2-methoxy-phenyl)-amide | | |
| 129 | 2-[5-(6-Methoxy-naphthalen-2-yl)-1 H- | 2 | 317 (M + H) |
| | indol-3-yl]-ethylamine | | 458 (M + CH3CN + H) |
| 130 | ′3-(2-Aminoethyl)-1-(3- | 56 | 412 (M + H) |
| | carbamoylpropyl)-5-(4- | | 453 (M + CH3CN + H) |
| | dibenzofuranyl)indole | | |
| 131 | ′3-(2-Aminoethyl)-5-(dibenzofuran-4- | 46 | 494 (M + H) |
| | yl)-1-(3-[N-(2,2,2- | | 534 (M + CH3CN + H) |
| | trifluoroethyl)carbamoyl]propyl)indole | | |
| 132 | ′3-(2-Aminoethyl)-1-[(4- | 64 | 461 (M + H) |
| | carboxyphenyl)methyl]-5-(4- | | 921 (2M + H) |
| | dibenzofuranyl)indole | | |
| | hydrochloride | | |
| 133 | ({2-[3-(2-Amino-ethyl)-5-dibenzofuran- | 79, 64 | 456 (M + H) |
| | 4-yl-indol-1-yl]-ethanoyl}-methyl- | | 911 (2M + H) |
| | amino)-acetic acid | | |
| 134 | 3-[3-(2-Amino-ethyl)-5-(4-cyano- | 28A/B | 426 (M + H) |
| | benzyloxy)-indol-1-ylmethyl]-benzoic | | |
| | acid trifluoroacetate | | |
| 135 | ({2-[3-(2-Amino-ethyl)-5-(3-cyano- | 28B | 421 (M + H) |
| | benzyloxy)-indol-1-yl]-ethanoyl}- | | |
| | methyl-amino)-acetic acid | | |
| 136 | 3-[3-(2-Amino-ethyl)-5-(4-carbamoyl- | 28B | 444 (M + H) |
| | benzyloxy)-indol-1-ylmethyl]-benzoic | | |
| | acid | | |
| 137 | 3-[3-(2-Amino-ethyl)-5-(4-carboxy- | 28B | 445 (M + H) |
| | benzyloxy)-indol-1-ylmethyl]-benzoic | | |
| | acid | | |
| 138 | 3-[3-(2--Amino-ethyl)-5-(3-carboxy- | 28B | 445 (M + H) |
| | benzyloxy)-indol-1-ylmethyl]-benzoic | | |
| | acid | | |
| 139 | [3-(2-Amino-ethyl)-5-(4-carbamoyl- | 28B | 368 (M + H) |
| | benzyloxy)-indol-1-yl]-acetic acid | | |
| 140 | 4-[3-(2-Amino-ethyl)-1-carboxymethyl- | 28B | 369 (M + H) |
| | 1H-indol-5-yloxymethyl]-benzoic acid | | |
| 141 | [3-(2-Amino-ethyl)-5-(3-carbamoyl- | 28B | 368 (M + H) |
| | benzyloxy)-indol-1-yl]-acetic acid | | |
| 142 | 3-[3-(2-Amino-ethyl)-1-carboxymethyl- | 28B | 369 (M + H) |
| | 1H-indol-5-yloxymethyl]-benzoic acid | | |
| 143 | 4-[3-(2-Amino-ethyl)-5-(3-carboxy- | 28B | 445 (M + H) |
| | benzyloxy)-indol-1-ylmethyl]-benzoic | | |
| | acid | | |
| 144 | 4-[3-(2-Amino-ethyl)-1-(3-cyano- | 28B | 426 (M + H) |
| | benzyl)-1H-indol-5-yloxymethyl]- | | |
| | benzoic acid | | |
| 145 | 5-[3-(2-Amino-ethyl)-5-(3-carbamoyl- | 28B | 434 (M + H) |
| | benzyloxy)-indol-1-ylmethyl]-furan-2- | | |
| | carboxylic acid | | |
| 146 | 5-[3-(2-Amino-ethyl)-5-(3-cyano- | 28B | 416 (M + H) |
| | benzyloxy)-indol-1-ylmethyl]-furan-2- | | |
| | carboxylic acid | | |
| 147 | 4-[3-(2-Amino-ethyl)-5-(4-carbamoyl- | 28B | 444 (M + H) |
| | benzyloxy)-indol-1-ylmethyl]-benzoic | | |
| | acid | | |
| 148 | 5-[3-(2-Amino-ethyl)-5-(4-carbamoyl- | 28B | 434 (M + H) |
| | benzyloxy)-indol-1-ylmethyl]-furan-2- | | |
| | carboxylic acid | | |
| 149 | [3-(2-Amino-ethyl)-5-(4-cyano- | 28B | 350 (M + H) |
| | benzyloxy)-indol-1-yl]-acetic acid | | |
| 150 | 5-[3-(2-Amino-ethyl)-5-(4-cyano- | 28B | 416 (M + H) |
| | benzyloxy)-indol-1-ylmethyl]-furan-2- | | |
| | carboxylic acid | | |
| 151 | 4-[3-(2-Amino-ethyl)-5-(3-carbamoyl- | 28B | 444 (M + H) |
| | benzyloxy)-indol-1-ylmethyl]-benzoic | | |
| | acid | | |
| 152 | 3-[3-(2-Amino-ethyl)-5-(3-carbamoyl- | 28B | 444 (M + H) |
| | benzyloxy)-indol-1-ylmethyl]-benzoic | | |
| | acid | | |
| 153 | 5-[3-(2-Amino-ethyl)-5-(3-carbamoyl- | 28B | 450 (M + H) |
| | benzyloxy)-indol-1-ylmethyl]- | | |
| | thiophene-2-carboxylic acid | | |
| 154 | 3-[3-(2-Amino-ethyl)-5-(3-cyano- | 28B | 426 (M + H) |
| | benzyloxy)-indol-1-ylmethyl]-benzoic | | |
| | acid | | |
| 155 | 5-[3-(2-Amino-ethyl)-5-(3-cyano- | 28B | 432 (M + H) |
| | benzyloxy)-indol-1-ylmethyl]- | | |
| | thiophene-2-carboxylic acid | | |
| 156 | 4-[3-(2-Amino-ethyl)-5-(biphenyl-4- | 28B | 477 (M + H) |
| | ylmethoxy)-indol-1-ylmethyl]-benzoic | | |
| | acid | | |
| 157 | 3-[3-(2-Amino-ethyl)-5-(biphenyl-4- | 28B | 477 (M + H) |
| | ylmethoxy)-indol-1-ylmethyl]-benzoic | | |
| | acid | | |
| 158 | 5-[3-(2-Amino-ethyl)-5-(biphenyl-4- | 28B | 467 (M + H) |
| | ylmethoxy)-indol-1-ylmethyl]-furan-2- | | |
| | carboxylic acid | | |
| 159 | 6-[3-(2-Amino-ethyl)-5-(2-chloro-4- | 28C | 454 (M + H) |
| | fluoro-benzyloxy)-indol-1-ylmethyl] | | |
| | nicotinic acid | | |
| 160 | 5-[3-(2-Amino-ethyl)-5-(2-chloro-4- | 28C | 459 (M + H) |
| | fluoro-benzyloxy)-indol-1-ylmethyl]- | | |
| | thiophene-2-carboxylic acid | | |
| 161 | 3-[3-(2-Amino-ethyl)-5-(2-chloro-4- | 28C | 453 (M + H) |
| | fluoro-benzyloxy)-indol-1-ylmethyl]- | | |
| | benzoic acid | | |
| 162 | 5-[3-(2-Amino-ethyl)-5-(2-chloro-4- | 28C | 443 (M + H) |
| | fluoro-benzyloxy)-indol-1-ylmethyl]- | | |
| | furan-2-carboxylic acid | | |
| 163 | 4-[3-(2-Amino-ethyl)-5-(2-chloro-4- | 28C | 453 (M + H) |
| | fluoro-benzyloxy)-indol-1-ylmethyl]- | | |
| | benzoic acid | | |
| 164 | (S)-1-{2-[3-(2-Amino-ethyl)-5-(2- | 28C | 474 (M + H) |
| | chloro-4-fluoro-benzyloxy)-indol-1-yl]- | | |
| | ethanoyl}-pyrrolidine-2-carboxylic acid | | |
| 165 | 6-[3-(2-Amino-ethyl)-5-(biphenyl-2- | 28C | 478 (M + H) |
| | ylmethoxy)-indol-1-ylmethyl]-nicotinic | | |
| | acid | | |
| 166 | 5-[3-(2-Amino-ethyl)-5-(biphenyl-2- | 28C | 483 (M + H) |
| | ylmethoxy)-indol-1-ylmethyl]- | | |
| | thiophene-2-carboxylic acid | | |
| 167 | 3-[3-(2-Amino-ethyl)-5-(biphenyl-2- | 28C | 477 (M + H) |
| | ylmethoxy)-indol-1-ylmethyl]-benzoic | | |
| | acid | | |
| 168 | 5-[3-(2-Amino-ethyl)-5-(biphenyl-2- | 28C | 467 (M + H) |
| | ylmethoxy)-indol-1-ylmethyl]-furan-2- | | |
| | carboxylic acid | | |
| 169 | 4-[3-(2-Amino-ethyl)-5-(biphenyl-2- | 28C | 477 (M + H) |
| | ylmethoxy)-indol-1-ylmethyl]-benzoic | | |
| | acid | | |
| 170 | 6-[3-(2-Amino-ethyl)-5-(2-benzyloxy- | 28C | 446 (M + H) |
| | ethoxy)-indol-1-ylmethyl]-nicotinic acid | | |
| 171 | 5-[3-(2-Amino-ethyl)-5-(2-benzyloxy- | 28C | 451 (M + H) |
| | ethoxy)-indol-1-ylmethyl]-thiophene-2- | | |
| | carboxylic acid | | |
| 172 | 3-[3-(2-Amino-ethyl)-5-(2-benzyloxy- | 28C | 445 (M + H) |
| | ethoxy)-indol-1-ylmethyl]-benzoic acid | | |
| 173 | 5-[3-(2-Amino-ethyl)-5-(2-benzyloxy- | 28C | 435 (M + H) |
| | ethoxy)-indol-1-ylmethyl]-furan-2- | | |
| | carboxylic acid | | |
| 174 | 4-[3-(2-Amino-ethyl)-5-(2-benzyloxy- | 28C | 445 (M + H) |
| | ethoxy)-indol-1-ylmethyl]-benzoic acid | | |
| 175 | 3-[3-(2-Amino-ethyl)-5-(2-fluoro- | 28C | 419 (M + H) |
| | benzyloxy)-indol-1-ylmethyl]-benzoic | | |
| | acid | | |
| 176 | 5-[3-(2-Amino-ethyl)-5-(2-fluoro- | 28C | 409 (M + H) |
| | benzyloxy)-indol-1-ylmethyl]-furan-2- | | |
| | carboxylic acid | | |
| 177 | 4-[3-(2-Amino-ethyl)-5-(2-fluoro- | 28C | 419 (M + H) |
| | benzyloxy)-indol-1-ylmethyl]-benzoic | | |
| | acid | | |
| 178 | 2-[3-(2-Amino-ethyl)-5-(2-fluoro- | 28C | 497 (M + H) |
| | benzyloxy)-indol-1-ylmethyl]-5-bromo- | | |
| | benzoic acid | | |
| 179 | 5-[3-(2-Amino-ethyl)-5-(3-nitro- | 28C | 452 (M + H) |
| | benzyloxy)-indol-1-ylmethyl]- | | |
| | thiophene-2-carboxylic acid | | |
| 180 | 3-[3-(2-Amino-ethyl)-5-(3-nitro- | 28C | 446 (M + H) |
| | benzyloxy)-indol-1-ylmethyl]-benzoic | | |
| | acid | | |
| 181 | 5-[3-(2-Amino-ethyl)-5-(3-nitro- | 28C | 436 (M + H) |
| | benzyloxy)-indol-1-ylmethyl]-furan-2- | | |
| | carboxylic acid | | |
| 182 | 6-[3-(2-Amino-ethyl)-5-(3-cyano- | 28C | 427 (M + H) |
| | benzyloxy)-indol-1-ylmethyl]-nicotinic | | |
| | acid | | |
| 183 | 1-{2-[3-(2-Amino-ethyl)-5-(3-cyano- | 28C | 447 (M + H) |
| | benzyloxy)-indol-1-yl]-ethanoyl}- | | |
| | pyrrolidine-2-carboxylic acid | | |
| 184 | 4-[3-(2-Amino-ethyl)-5-(6-chloro- | 28C | 479 (M + H) |
| | benzo[1,3]dioxol-5-ylmethoxy)-indol-1- | | |
| | ylmethyl]-benzoic acid | | |
| 185 | 6-[3-(2-Amino-ethyl)-5-(3-methoxy- | 28C | 432 (M + H) |
| | benzyloxy)-indol-1-ylmethyl]-nicotinic | | |
| | acid | | |
| 186 | 5-[3-(2-Amino-ethyl)-5-(3-methoxy- | 28C | 421 (M + H) |
| | benzyloxy)-indol-1-ylmethyl]-furan-2- | | |
| | carboxylic acid | | |
| 187 | 4-[3-(2-Amino-ethyl)-5-(3-methoxy- | 28C | 431 (M + H) |
| | benzyloxy)-indol-1-ylmethyl]-benzoic | | |
| | acid | | |
| 188 | 6-[3-(2-Amino-ethyl)-5-(6-fluoro-4H- | 28C | 478 (M + H) |
| | benzo[1,3]dioxin-8-ylmethoxy)-indol-1- | | |
| | ylmethyl]-nicotinic acid | | |
| 189 | 5-[3-(2-Amino-ethyl)-5-(6-fluoro-4H- | 28C | 483 (M + H) |
| | benzo[1,3]dioxin-8-ylmethoxy)-indol-1- | | |
| | ylmethyl]-thiophene-2-carboxylic acid | | |
| 190 | 3-[3-(2-Amino-ethyl)-5-(6-fluoro-4H- | 28C | 477 (M + H) |
| | benzo[1,3]dioxin-8-ylmethoxy)-indol-1- | | |
| | ylmethyl]-benzoic acid | | |
| 191 | 5-[3-(2-Amino-ethyl)-5-(6-fluoro-4H- | 28C | 467 (M + H) |
| | benzo[1,3]dioxin-8-ylmethoxy)-indol-1- | | |
| | ylmethyl]-furan-2-carboxylic acid | | |
| 192 | 4-[3-(2-Amino-ethyl)-5-(6-fluoro-4H- | 28C | 477 (M + H) |
| | benzo[1,3]dioxin-8-ylmethoxy)-indol-1- | | |
| | ylmethyl]-benzoic acid | | |
| 193 | 2-[3-(2-Amino-ethyl)-5-(6-fluoro-4H- | 28C | 555 (M + H) |
| | benzo[1,3]dioxin-8-ylmethoxy)-indol-1- | | |
| | ylmethyl]-5-bromo-benzoic acid | | |
| 194 | 1-{2-[3-(2-Amino-ethyl)-5-(6-fluoro- | 28C | 498 (M + H) |
| | 4H-benzo[1,3]dioxin-8-ylmethoxy)- | | |
| | indol-1-yl]-ethanoyl}-pyrrolidine-2- | | |
| | carboxylic acid | | |
| 195 | 5-[3-(2-Amino-ethyl)-5-(3-cyano- | 28C | 432 (M + H) |
| | benzyloxy)-indol-1-ylmethyl]- | | |
| | thiophene-2-carboxylic acid | | |
| 196 | 3-[3-(2-Amino-ethyl)-5-(3-cyano- | 28C | 426 (M + H) |
| | benzyloxy)-indol-1-ylmethyl]-benzoic | | |
| | acid | | |
| 197 | 6-[3-(2-Amino-ethyl)-5-(3-nitro- | 28C | 447 (M + H) |
| | benzyloxy)-indol-1-ylmethyl]-nicotinic | | |
| | acid | | |
| 198 | 2-[3-(2-Amino-ethyl)-5-(3-nitro- | 28C | 424 (M + H) |
| | benzyloxy)-indol-1-ylmethyl]-5-bromo- | | |
| | benzoic acid | | |
| 199 | (S)-1-{2-[3-(2-Amino-ethyl)-5- | 28C | 422 (M + H) |
| | benzyloxy-indol-1-yl]-ethanoyl}- | | |
| | pyrrolidine-2-carboxylic acid | | |
| 200 | 2-[3-(2-Amino-ethyl)-5-(3-cyano- | 28C | 504 (M + H) |
| | benzyloxy)-indol-1-ylmethyl]-5-bromo- | | |
| | benzoic acid | | |
| 201 | 5-[3-(2-Amino-ethyl)-5-(6-chloro- | 28C | 485 (M + H) |
| | benzo[1,3]dioxol-5-ylmethoxy)-indol-1- | | |
| | ylmethyl]-thiophene-2-carboxyic acid | | |
| 202 | 3-[3-(2-Amino-ethyl)-5-(3-methoxy- | 28C | 431 (M + H) |
| | benzyloxy)-indol-1-ylmethyl]-benzoic | | |
| | acid | | |
| 203 | 2-[3-(2-Amino-ethyl)-5-(6-chloro- | 28C | 557 (M + H) |
| | benzo[1,3]dioxol-5-ylmethoxy)-indol-1- | | |
| | ylmethyl]-5-bromo-benzoic acid | | |
| 204 | 5-[3-(2-Amino-ethyl)-5-benzyloxy- | 28C | 391 (M + H) |
| | indol-1-ylmethyl]-furan-2-carboxylic | | |
| | acid | | |
| 205 | 5-[3-(2-Amino-ethyl)-5-(3-carbamoyl- | 28C | 522 (M + H) |
| | benzyloxy)-indol-1-ylmethyl]-2-bromo- | | |
| | benzoic acid | | |
| 206 | 3-[3-(2-Amino-ethyl)-5-(6-chloro- | 28C | 479 (M + H) |
| | benzo[1,3]dioxol-5-ylmethoxy)-indol-1- | | |
| | ylmethyl]-benzoic acid | | |
| 207 | 5-[3-(2-Amino-ethyl)-5-(6-chloro- | 28C | 469 (M + H) |
| | benzo[1,3]dioxol-5-ylmethoxy)-indol-1- | | |
| | ylmethyl]-furan-2-carboxylic | | |

Formulations for pharmaceutical use incorporating compounds of the present invention can be prepared in various forms and with numerous excipients. Examples of such formulations are given below:

### EXAMPLE 208

Inhalant Formulation

A compound of Formula (I), (1 mg to 100 mg) is aerosolized from a metered dose inhaler to deliver the desired amount of drug per use.

### EXAMPLE 209

Tablet Formulation

| Tablets/Ingredients | Per Tablet |
|---|---|
| 1. Active ingredient | 40 mg |
| (Cpd of Form. (I) | |
| 2. Corn Starch | 20 mg |
| 3. Alginic acid | 20 mg |
| 4. Sodium Alginate | 20 mg |
| 5. Mg stearate | 1.3 mg |

Procedure for tablet formulation:

Ingredients 1, 2, 3 and 4 are blended in a suitable mixer/blender. Sufficient water is added portion-wise to the blend with careful mixing after each addition until the mass is of a consistency to permit its conversion to wet granules. The wet mass is converted to granules by passing it through an oscillating granulator using a No. 8 mesh (2.38 mm) screen. The wet granules are then dried in an oven at 140° F. (60° C.) until dry. The dry granules are lubricated with ingredient No. 5, and the lubricated granules are compressed on a suitable tablet press.

### EXAMPLE 210

Parenteral Formulation

A pharmaceutical composition for parenteral administration is prepared by dissolving an appropriate amount of a compound of Formula I in polyethylene glycol with heating. This solution is then diluted with water for injections (to 100 mL). The solution is then rendered sterile by filtration through a 0.22 micron membrane filter and sealed in sterile containers.

All publications, including but not limited to patents and patent applications cited in this specification are herein incorporated by reference as if each individual publication were specifically and individually indicated to be incorporated by reference as though fully set forth.

What is claimed is:

1. A compound according to formula (I) hereinbelow: 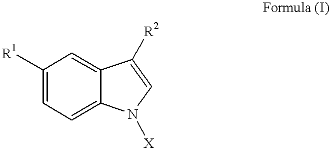
wherein R¹ is aryl;
R² is C₁₋₄ NHR, wherein R is H or C(NH)NH₂;
X is H or SO₂R, wherein R is selected from the group consisting of C₁₋₂₀ alkyl or aryl.

2. A compound according to claim 1 having a structure according to formula (II): 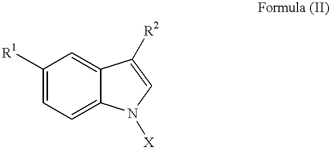
wherein R¹ represents aryl;
R² represents C₁₋₄ NHR, wherein R is H or C(NH)NH₂; and
X represents SO₂R, wherein R is selected from the group consisting of C₁₋₂₀ alkyl or aryl.

3. A compound according to claim 1 having the structure according to formula (III) hereinbelow: 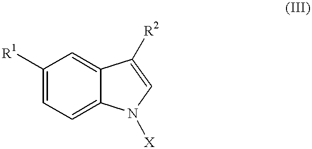
wherein:
R¹ represents ArCH₂O, wherein Ar represents aryl;
R₂ represents (C₁₋₄)NH₂; and
X represents CH₂R, wherein R is selected from the group consisting of C₁₋₂₀ alkyl, aryl and C(O)NR′R″, wherein R′ and R″ are, independently, H or C₁₋₁₀ alkyl or aryl.

4. A compound according to claim 1 having the structure according to formula (IV) hereinbelow: 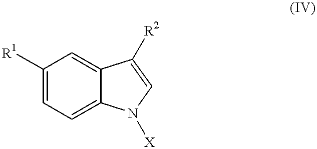
wherein R¹ represents ArCH₂O, wherein Ar represents aryl;
R² represents (C₁₋₄)NH₂; and
X represents SO₂R, wherein R is C₁₋₂₀ alkyl or aryl.

5. A compound according to claim 4 according to formula (V) hereinbelow: 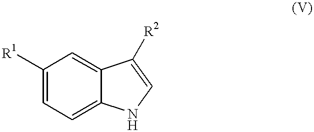
wherein:
R¹ represents aryl; and R² represents (C₁₋₄)NH₂.

6. A compound according to claim 1 having a structure according to formula (VI) hereinbelow: 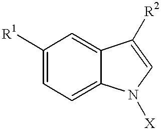
wherein R¹ represents aryl;
R² represents (C₁₋₄)NH₂; and
X represents CH₂R, wherein R represents C₁₋₂₀ alkyl, aryl or C(O)NR′R″, wherein R′ and R″ are, independently, H, C₁₋₁₂ alkyl or C₅₋₁₂ aryl.

7. A compound according to claim 1 having a structure according to formula (VII) hereinbelow: 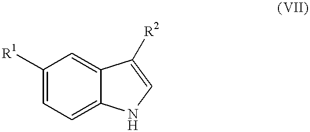
wherein:
R¹ represents ArCH₂O; and
R² represents (C₁₋₄)NH₂.

8. A compound according to claim 1 selected from the group consisting of 1-phenylsulfonyl-3-(2-aminoethyl)-5-(1-naphthyl)indole hydrochloride;
1-(2-naphthylsulfonyl)-3-(2-guanidinoethyl)-5-(1-naphthyl)indole hydrochloride;
1-phenylsulfonyl-3-(2-aminoethyl)-5-(2-thienyl)indole hydrochloride;
1-phenylsulfonyl-3-(2-aminoethyl)-5-(3-trifluoromethylphenyl)indole hydrochloride;
3-[(2-naphthyl)sulfonyl]-3-(2-aminoethyl)-5-(1-naphthyl)indole hydrochloride;
1-phenylsulfonyl-3-(2-aminoethyl)-5-(2-naphthyl)indole hydrochloride;
1-phenylsulfonyl-3-(2-aminoethyl)-5-phen ylindole hydrochloride;
1-phenylsulfonyl-3-(2-aminoethyl)-5-(benzothiophen-2-yl) indole hydrochloride;
1-phenylsulfonyl-3-aminomethyl-5-(1-naphthyl)indole trifluoroacetate;
4-[1-phenylsulfonyl-3-(2-aminoethyl)indol-5-yl]benzophenone;
2-[1-Benzenesulfonyl-5-(4-methyl-naphthalen-1-yl)-1-H-indol-3-yl]-ethylamine;
4-[3-(2-Amino-ethyl)-1-benzenesulfonyl-1-H-indol-5-yl]-naphthalen-1-ylamine;
2-[1-Benzenesulfonyl-5-(2-methoxymethyl-naphthalen-1-yl)-1-H-indol-3-yl]-ethylamine;
6-[3-(2-Amino-ethyl)-1-benzenesulfonyl-1-H-indol-5-yl]-naphthalen-2-ol;
1-[(3-carbamoylphenyl)methyl]-3-(2-anoethyl)-5-(4-dibenzofuranyl)indole hydrochloride;
1-(5-cyanopentyl)-3-(2-aminoethyl)-5-(4-dibenzofuranyl)indole hydrochloride;
1-(2-carbamoylethyl)-3-(2-aminoethyl)-5-(4-dibenzofuranyl)indole hydrochloride;
1-[(3-carboxyphenyl)methyl]-3-(2-aminoethyl)-5-(1-naphthyl)indole hydrochloride;
3-(2-aminoethyl)-5-(4-dibenzofuranyl)indole-1-pentanoic acid hydrochloride;
1-[(3-carbamoylphenyl)methyl]-3-(2-aminoethyl)-5-(4-dibenzofuranyl)indole hydrochloride;
1-(5-cyanopentyl)-3-(2-aminoethyl)-5-(4-dibenzofuranyl)indole hydrochloride;
1-(2-carbamoylethyl)-3-(2-aminoethyl)-5-(4-dibenzofuranyl)indole hydrochloride;
1-[(3-carboxyphenyl)methyl]-3-(2-aminoethyl)-5-(1-naphthyl)indole hydrochloride;
3-(2-aminoethyl)-5-(4-dibenzofuranyl)indole-1-pentanoic acid hydrochloride;
1-(carbamoylmethyl)-3-(2-aminoethyl)-5-(2-naphthyl)indole trifluoroacetate;
1-(N-methylcarbamoylmethyl)-3-(2-aminoethyl)-5-(1-naphthyl)indole trifluoroacetate;
1-[(3-carbamoylphenyl)methyl]-3-(2-aminoethyl)-5-(1-naphthyl)indole hydrochloride;
1-[(3-morpholinoylphenyl)methyl]-3-(2-aminoethyl)-5-(1-naphthyl)indole hydrochloride;
1-[(3-(3-trifluoromethylbenzyl)carbamoylphenyl)methyl]-3-(2-aminoethyl)-5-(1-naphthyl)indole hydrochloride;
1-[(4-carbamoylphenyl)methyl]-3-(2-aminoethyl)-5-(4-dibenzofuranyl)indole hydrochloride;
1-(carbamoylmethyl)-3-(2-aminoethyl)-5-(4-dibenzofuranyl)indole hydrochloride;
1-(4-carbomethoxyphenyl)methyl-3-(2-aminoethyl)-5-(4-dibenzofuranyl) indole hydrochloride;
1-(3-carbomethoxyphenyl)methyl-3-(2-aminoethyl)-5-(4-dibenzofuranyl) indole hydrochloride;
1-(2-cyanophenyl)methyl-3-(2-aminoethyl)-5-(4-dibenzofuranyl)indole hydrochloride;
1-(3-cyanophenyl)methyl-3-(2-aminoethyl)-5-(4-dibenzofuranyl)indole hydrochloride;
1-(4-acetamidophenyl)methyl-3-(2-aminoethyl)-5-(4-dibenzofuranyl)indole hydrochloride;
1-(4-cyanobutyl)-3-(2-aminoethyl)-5-(4-dibenzofuranyl)indole hydrochloride;
1-[(2-carbamoylphenyl)methyl]-3-(2-aminoethyl)-5-(4-dibenzofuranyl)indole hydrochloride;
1-ethyl-3-(2-aminoethyl)-5-(4-dibenzofuranyl)indole hydrochloride;
1-[(3-carboxyphenyl)methyl]-3-(2-aminoethyl)-5-(benzothiophen-2-yl)indole hydrochloride;
1-[(4-carboxyphenyl)methyl]-3-(2-aminoethyl)-5-(benzothiophen-2-yl)indole hydrochloride;
1-[(3-carboxyphenyl)methyl]-3-(2-aminoethyl)-5-(4-diboenzofuranyl)indole hydrochloride;
3-(2-aminoethyl)-5-(4-dibenzofuranyl)indole-1-butanoic acid hydrochloride;
1-[(N-(3-trifluoromethyphenyl)methyl)acetamido]-3-(2-aminoethyl)-5-(1-naphthyl)indole;
1-[(N-(4-pyridyl)methyl)acetamido]-3-(2-aminoethyl)-5-(1-naphthyl)indole;
1-[(N-(3-methoxyphenyl)methyl)acetamido]-3-(2-aminoethyl)-5-(1-naphthyl)indole;
1-[(N-(4-sulfonamidophenyl)methyl)acetamido]-3-(2-aminoethyl)-5-(1-naphthyl)indole;
1-[(N-benzyl)acetamido]-3-(2-aminoethyl)-5-(1-naphthyl)indole;
1-[(N-(2,5-difluorophenyl)methyl)acetamido]-3-(2-aminoethyl)-5-(1-naphthyl)indole;
1-[(N-(2,4-dichlorophenyl)methyl)acetamido]-3-(2-aminoethyl)-5-(1-naphthyl)indole;
1-[(N-(2-benzimidazole)methyl)acetamido]-3-(2-aminoethyl)-5-(1-naphthyl)indole;
1-[N-(3-pyridyl)acetamido]-3-(2-aminoethyl)-5-(1-naphthyl)indole;
1-[N-(2-thiazole)acetamido]-3-(2-aminoethyl)-5-(1-naphthyl)indole;
1-[(N-(2-thiophene)methyl)acetamido]-3-(2-aminoethyl)-5-(1-naphthyl)indole;
1-[trans-(N-cyclopropylphenyl)acetamido]-3-(2-aminoethyl)-5-(1-naphthyl)indole;
1-[N-(4-carboethoxy)piperidine)acetamido]-3-(2-aminoethyl)-5-(1-naphthyl)indole;
1-[N-(3-methoxyphenyl)acetamido]-3-(2-aminoethyl)-5-(4-dibenzofuranyl)indole;
3-(2-aminoethyl)-5-(2-naphthyl)indole hydrochloride;
3-(2-aminoethyl)-5-(1-naphthyl)indole hydrochloride;
3-(2-aminoethyl)-5-(benzofuran-2-yl)indole hydrochloride;
3-(3-aminopropyl)-5-(1-naphthyl)indole trifluoroacetate;
3-(2-aminoethyl)-5-(4-dibenzofuranyl)indole hydrochloride;
3-(2-aminoethyl)-5-(2-thienyl)indole hydrochloride;
3-(2-aminoethyl)-5-(3-trifluoromethylphenyl)indole hydrochloride;
4-[3-(2-aminoethyl)indol-5-yl]benzophenone hydrochloride;
3-(2-aminoethyl)-5-(benzothiophen-2-yl)indole hydrochloride;
2-[5-(4-Methyl-naphthalen-1-yl)-1 H-indol-3-yl]-ethylamine;
6-[3-(2-Amino-ethyl)-1 H-indol-5-yl]-naphthalen-2-ol;
7-[3-(2-Amino-ethyl)-1 H-indol-5-yl]-3-hydroxy-naphthalene-2-carboxylic acid (2-methoxy-phenyl)-amide;
2-[5-(6-Methoxy-naphthalen-2-yl)-1 H-indol-3-yl]-ethylamine;
1-(4-methoxy)benzenesulfonyl-3-(2-aminoethyl)-5-(1-naphthyl) methyloxyindole trifluoroacetate;
1-(4-methoxy)benzenesulfonyl-3-(2-aminoethyl)-5-(2-naphthyl) methyloxyindole trifluoroacetate;
1-(8-quinoline)sulfonyl-3-(2-aminoethyl)-5-(2-naphthyl)methyloxyindole trifluoroacetate;
1-(2-thienyl)sulfonyl-3-(2-aminoethyl)-5-(4-biphenyl)methyloxyindole trifluoroacetate;
1-(2-chloro-4-fluoro)benzenesulfonyl-3-(2-aminoethyl)-5-(2-biphenyl)methyloxyindole trifluoroacetate;
1-(3-chloro-4-fluoro)benzenesulfonyl-3-(2-aminoethyl)-5-(2-naphthyl) methyloxyindole trifluoroacetate;
1-(8-quinoline)sulfonyl-3-(2-aminoethyl)-5-(2-biphenyl)methyloxyindole trifluoroacetate;
1-(4-methoxy)benzenesulfonyl-3-(2-aminoethyl)-5-(4-biphenyl) methyloxyindole trifluoroacetate;
1-(2-thienyl)sulfonyl-3-(2-aminoethyl)-5-[(4-tert-butyl)phenyl] methyloxyindole trifluoroacetate;
3-(2-aminoethyl)-5-[(3-phenoxy)benzyloxy]indole hydrochloride;
3-(2-aminoethyl)-5-[(2-naphthyl)methyloxy]indole hydrochloride;
3-(2-aminoethyl)-5-[(2-phenyl)benzyloxy]indole hydrochloride;
3-(2-aminoethyl)-5-[(4-phenyl)benzyloxy]indole hydrochloride;
1-(N-Carboxmethyl-N-methylcarbamoylmethyl)-3-(2-aminoethyl)-5-(4-dibenzofuranyl)indole trifluoroacetate;
3-[3-(2-Amino-ethyl)-5-(4-cyano-benzyloxy)-indol-1-ylmethyl]-benzoic acid trifluoroacetate;
({2-[3-(2-Amino-ethyl)-5-(3-cyano-benzyloxy)-indol-1-yl]-ethanoyl}-methyl-amino)-acetic acid;
3-[3-(2-Amino-ethyl)-5-(4-carbamoyl-benzyloxy)-indol-1-ylmethyl]-benzoic acid;
3-[3-(2-Amino-ethyl)-5-(4-carboxy-benzyloxy)-indol-1-ylmethyl]-benzoic acid;
3-[3-(2-Amino-ethyl)-5-(3-carboxy-benzyloxy)-indol-1-ylmethyl]-benzoic acid;
[3-(2-Amino-ethyl)-5-(4-carbamoyl-benzyloxy)-indol-1-yl]-acetic acid;
4-[3-(2-Amino-ethyl)-1-carboxymethyl-1H-indol-5-yloxymethyl]-benzoic acid;
[3-(2-Amino-ethyl)-5-(3-carbamoyl-benzyloxy)-indol-1-yl]-acetic acid;
3-[3-(2-Amino-ethyl)-1-carboxymethyl-1H-indol-5-yloxymethyl]-benzoic acid;
4-[3-(2-Amino-ethyl)-5-(3-carboxy-benzyloxy)-indol-1-ylmethyl]-benzoic acid;
4-[3-(2-Amino-ethyl)-1-(3-cyano-benzyl)-1H-indol-5-yloxymethyl]-benzoic acid;
5-[3-(2-Amino-ethyl)-5-(3-carbamoyl-benzyloxy)-indol-1-ylmethyl]-furan-2-carboxylic acid;
5-[3-(2-Amino-ethyl)-5-(3-cyano-benzyloxy)-indol-1-ylmethyl]-furan-2-carboxylic acid;
4-[3-(2-Amino-ethyl)-5-(4-carbamoyl-benzyloxy)-indol-1-ylmethyl]-benzoic acid;
5-[3-(2-Amino-ethyl)-5-(4-carbamoyl-benzyloxy)-indol-1-ylmethyl]-furan-2-carboxylic acid;
[3-(2-Amino-ethyl)-5-(4-cyano-benzyloxy)-indol-1-yl]-acetic acid;
5-[3-(2-Amino-ethyl)-5-(4-cyano-benzyloxy)-indol-1-ylmethyl]-furan-2-carboxylic acid;
4-[3-(2-Amino-ethyl)-5-(3-carbamoyl-benzyloxy)-indol-1-ylmethyl]-benzoic acid;
3-[3-(2-Amino-ethyl)-5-(3-carbamoyl-benzyloxy)-indol-1-ylmethyl]-benzoic acid;
5-[3-(2-Amino-ethyl)-5-(3-carbamoyl-benzyloxy)-indol-1-ylmethyl]-thiophene-2-carboxylic acid;
3-[3-(2-Amino-ethyl)-5-(3-cyano-benzyloxy)-indol-1-ylmethyl]-benzoic acid;
5-[3-(2-Amino-ethyl)-5-(3-cyano-benzyloxy)-indol-1-ylmethyl]-thiophene-2-carboxylic acid;
4-[3-(2-Amino-ethyl)-5-(biphenyl-4-ylmethoxy)-indol-1-ylmethyl]-benzoic acid;
3-[3-(2-Amino-ethyl)-5-(biphenyl-4-ylmethoxy)-indol-1-ylmethyl]-benzoic acid;
5-[3-(2-Amino-ethyl)-5-(biphenyl-4-ylmethoxy)-indol-1-ylmethyl]-furan-2-carboxylic acid;
6-[3-(2-Amino-ethyl)-5-(2-chloro-4-fluoro-benzyloxy)-indol-1-ylmethyl]-nicotinic acid;
5-[3-(2-Amino-ethyl)-5-(2-chloro-4-fluoro-benzyloxy)-indol-1-ylmethyl]-thiophene-2-carboxylic acid;
3-[3-(2-Amino-ethyl)-5-(2-chloro-4-fluoro-benzyloxy)-indol-1-ylmethyl]-benzoic acid;
5-[3-(2-Amino-ethyl)-5-(2-chloro-4-fluoro-benzyloxy)-indol-1-ylmethyl]-furan-2-carboxylic acid;
4-[3-(2-Amino-ethyl)-5-(2-chloro-4-fluoro-benzyloxy)-indol-1-ylmethyl]-benzoic acid;
(S)-1-{2-[3-(2-Amino-ethyl)-5-(2-chloro-4-fluoro-benzyloxy)-indol-1-yl]-ethanoyl}-pyrrolidine-2-carboxylic acid;
6-[3-(2-Amino-ethyl)-5-(biphenyl-2-ylmethoxy)-indol-1-ylmethyl]-nicotinic acid;
5-[3-(2-Amino-ethyl)-5-(biphenyl-2-ylmethoxy)-indol-1-ylmethyl]-thiophene-2-carboxylic acid;
3-[3-(2-Amino-ethyl)-5-(biphenyl-2-ylmethoxy)-indol-1-ylmethyl]-benzoic acid;
5-[3-(2-Amino-ethyl)-5-(biphenyl-2-ylmethoxy)-indol-1-ylmethyl]-furan-2-carboxylic acid;
4-[3-(2-Amino-ethyl)-5-(biphenyl-2-ylmethoxy)-indol-1-ylmethyl]-benzoic acid;
6-[3-(2-Amino-ethyl)-5-(2-benzyloxy-ethoxy)-indol-1-ylmethyl]-nicotinic acid;
5-[3-(2-Amino-ethyl)-5-(2-benzyloxy-ethoxy)-indol-1-ylmethyl]-thiophene-2-carboxylic acid;
3-[3-(2-Amino-ethyl)-5-(2-benzyloxy-ethoxy)-indol-1-ylmethyl]-benzoic acid;
5-[3-(2-Amino-ethyl)-5-(2-benzyloxy-ethoxy)-indol-1-ylmethyl]-furan-2-carboxylic acid;
4-[3-(2-Amino-ethyl)-5-(2-benzyloxy-ethoxy)-indol-1-ylmethyl]-benzoic acid;
3-[3-(2-Amino-ethyl)-5-(2-fluoro-benzyloxy)-indol-1-ylmethyl]-benzoic acid;
5-[3-(2-Amino-ethyl)-5-(2-fluoro-benzyloxy)-indol-1-ylmethyl]-furan-2-carboxylic acid;
4-[3-(2-Amino-ethyl)-5-(2-fluoro-benzyloxy)-indol-1-ylmethyl]-benzoic acid;
2-[3-(2-Amino-ethyl)-5-(2-fluoro-benzyloxy)-indol-1-ylmethyl]-5-bromo-benzoic acid;
5-[3-(2-Amino-ethyl)-5-(3-nitro-benzyloxy)-indol-1-ylmethyl]-thiophene-2-carboxylic acid;
3-[3-(2-Amino-ethyl)-5-(3-nitro-benzyloxy)-indol-1-ylmethyl]-benzoic acid;
5-[3-(2-Amino-ethyl)-5-(3-nitro-benzyloxy)-indol-1-ylmethyl]-furan-2-carboxylic acid;
6-[3-(2-Amino-ethyl)-5-(3-cyano-benzyloxy)-indol-1-ylmethyl]-nicotinic acid;
1-{2-[3-(2-Amino-ethyl)-5-(3-cyano-benzyloxy)-indol-1-yl]-ethanoyl}-pyrrolidine-2-carboxylic acid;
4-[3-(2-Amino-ethyl)-5-(6-chloro-benzo[1,3]dioxol-5-ylmethoxy)-indol-1-ylmethyl]-benzoic acid;
6-[3-(2-Amino-ethyl)-5-(3-methoxy-benzyloxy)-indol-1-ylmethyl]-nicotinic acid;
5-[3-(2-Amino-ethyl)-5-(3-methoxy-benzyloxy)-indol-1-ylmethyl]-furan-2-carboxylic acid;
4-[3-(2-Amino-ethyl)-5-(3-methoxy-benzyloxy)-indol-1-ylmethyl]-benzoic acid;
6-[3-(2-Amino-ethyl)-5-(6-fluoro-4H-benzo[1,3]dioxin-8-ylmethoxy)-indol-1-ylmethyl]-nicotinic acid;
5-[3-(2-Amino-ethyl)-5-(6-fluoro-4H-benzo[1,3]dioxin-8-ylmethoxy)-indol-1-ylmethyl]-thiophene-2-carboxylic acid;
3-[3-(2-Amino-ethyl)-5-(6-fluoro-4H-benzo[1,3]dioxin-8-ylmethoxy)-indol-1-ylmethyl]-benzoic acid;
5-[3-(2-Amino-ethyl)-5-(6-fluoro-4H-benzo[1,3]dioxin-8-ylmethoxy)-indol-1-ylmethyl]-furan-2-carboxylic acid;
4-[3-(2-Amino-ethyl)-5-(6-fluoro-4H-benzo[1,3]dioxin-8-ylmethoxy)-indol-1-ylmethyl]-benzoic acid;
2-[3-(2-Amino-ethyl)-5-(6-fluoro-4H-benzo[1,3]dioxin-8-ylmethoxy)-indol-1-ylmethyl]-5-bromo-benzoic acid;
1-{2-[3-(2-Amino-ethyl)-5-(6-fluoro-4H-benzo[1,3]dioxin-8-ylmethoxy)-indol-1-yl]-ethanoyl}-pyrrolidine-2-carboxylic acid;
5-[3-(2-Amino-ethyl)-5-(3-cyano-benzyloxy)-indol-1-ylmethyl]-thiophene-2-carboxylic acid;
3-[3-(2-Amino-ethyl)-5-(3-cyano-benzyloxy)-indol-1-ylmethyl]-benzoic acid;
6-[3-(2-Amino-ethyl)-5-(3-nitro-benzyloxy)-indol-1-ylmethyl]-nicotinic acid;
2-[3-(2-Amino-ethyl)-5-(3-nitro-benzyloxy)-indol-1-ylmethyl]-5-bromo-benzoic acid;
(S)-1-{2-[3-(2-Amino-ethyl)-5-benzyloxy-indol-1-yl]-ethanoyl}-pyrrolidine-2-carboxylic acid;
2-[3-(2-Amino-ethyl)-5-(3-cyano-benzyloxy)-indol-1-ylmethyl]-5-bromo-benzoic acid;
5-[3-(2-Amino-ethyl)-5-(6-chloro-benzo[1,3]dioxol-5-ylmethoxy)-indol-1-ylmethyl]-thiophene-2-carboxyic acid;
3-[3-(2-Amino-ethyl)-5-(3-methoxy-benzyloxy)-indol-1-ylmethyl]-benzoic acid;
2-[3-(2-Amino-ethyl)-5-(6-chloro-benzo[1,3]dioxol-5-ylmethoxy)-indol-1-ylmethyl]-5-bromo-benzoic acid;
5-[3-(2-Amino-ethyl)-5-benzyloxy-indol-1-ylmethyl]-furan-2-carboxylic acid;
5-[3-(2-Amino-ethyl)-5-(3-carbamoyl-benzyloxy)-indol-1-ylmethyl]-2-bromo-benzoic acid;
3-[3-(2-Amino-ethyl)-5-(6-chloro-benzo[1,3]dioxol-5-ylmethoxy)-indol-1-ylmethyl]-benzoic acid;
5-[3-(2-Amino-ethyl)-5-(6-chloro-benzo[1,3]dioxol-5-ylmethoxy)-indol-1-ylmethyl]-furan-2-carboxylic acid;
1-(4-trifluoromethylphenyl)methyl-3-(2-aminoethyl)-5-(2-naphthyl)methyloxyindole trifluoroacetate;
1-(3-trifluoromethylphenyl)methyl-3-(2-aminoethyl)-5-(4-cyanophenyl) methyloxyindole trifluoroacetate;
1-(3,4-dichlorophenyl)methyl-3-(2-aminoethyl)-5-(4-cyanophenyl) methyloxyindole trifluoroacetate;
1-(3,4-dichlorophenyl)methyl-3-(2-aminoethyl)-5-(3-cyanophenyl) methyloxyindole trifluoroacetate;
1-[(3,5-bis-trifluoromethyl)phenylmethyl-3-(2-aminoethyl)-5-(3-cyanophenyl)methyloxyindole trifluoroacetate;
1-[(3,5-bis-trifluoromethyl)phenyl]methyl-3-(2-aminoethyl)-5-(4-cyanophenyl)methyloxyindole trifluoroacetate; and
1-(4-trifluoromethylphenyl)methyl-3-(2-aminoethyl)-5-(3-cyanophenyl) methyloxyindole trifluoroacetate;
or a pharmaceutically acceptable salt thereof.

9. A compound according to claim 2 selected from the group consisting of:
1-phenylsulfonyl-3-(2-aminoethyl)-5-(1-naphthyl)indole hydrochloride;
1-(2-naphthylsulfonyl)-3-(2-guanidinoethyl)-5-(1-naphthyl)indole hydrochloride;
1-phenylsulfonyl-3-(2-aminoethyl)-5-(2-thienyl)indole hydrochloride;
1-phenylsulfonyl-3-(2-aminoethyl)-5-(3-trifluoromethylphenyl)indole hydrochloride;
1-(2-naphthyl)sulfonyl]-3-(2-aminoethyl)-5-(1-naphthyl)indole hydrochloride;
1-phenylsulfonyl-3-(2-aminoethyl)-5-(2-naphthyl)indole hydrochloride;
1-phenylsulfonyl-3-(2-aminoethyl)-5-phenylindole hydrochloride;
1-phenylsulfonyl-3-(2-aminoethyl)-5-(benzothiophen-2-yl)indole hydrochloride;
1-phenylsulfonyl-3-aminomethyl-5-(1-naphthyl)indole trifluoroacetate;
4-[-phenylsulfonyl-3-(2-aminoethyl)indol-5-yl]benzophenone;
2-[1-Benzenesulfonyl-5-(4-methyl-naphthalen-1-yl)-1-H-indol-3-yl]-ethylamine;
4-[3-(2-Amino-ethyl)-l-benzenesulfonyl-1-H-indol-5-yl]-naphthalen-1-ylamine;
2-[1-Benzenesulfonyl-5-(2-methoxymethyl-naphthalen-1-yl)-1-H-indol-3-yl]-ethylamine; and
6-[3-(2-Amino-ethyl)-1-benzenesulfonyl-1-H-indol-5-yl]-naphthalen-2-ol;
or a pharmaceutically acceptable salt thereof.

10. A compound according to claim 3 selected from the group consisting of:
1-(4-trifluoromethylphenyl)methyl-3-(2-aminoethyl)-5-(2-naphthyl)methyloxyindole trifluoroacetate;
1-(3-trifluoromethylphenyl)methyl-3-(2-aminoethyl)-5-(4-cyanophenyl) methyloxyindole trifluoroacetate;
1-(3,4-dichlorophenyl)methyl-3-(2-aminoethyl)-5-(4-cyanophenyl) methyloxyindole trifluoroacetate;
1-(3,4-dichlorophenyl)methyl-3-(2-aminoethyl)-5-(3-cyanophenyl) methyloxyindole trifluoroacetate;
1-[(3,5-bis-trifluoromethyl)phenyl]methyl-3-(2-aminoethyl)-5-(3-cyanophenyl)methyloxyindole trifluoroacetate;
1-[(3,5-bis-trifluoromethyl)phenyl]methyl-3-(2-aminoethyl)-5-(4-cyanophenyl)methyloxyindole trifluoroacetate;
1-(4-trifluoromethylphenyl)methyl-3-(2-aminoethyl)-5-(3-cyanophenyl) methyloxyindole trifluoroacetate;
or a pharmaceutically acceptable salt thereof.

11. Acompound according to claim 4 selected from the group consisting of:
1-(4-methoxy)benzenesulfonyl-3-(2-aminoethyl)-5-(1-naphthyl) methyloxyindole trifluoroacetate;
1-(4-methoxy)benzenesulfonyl-3-(2-aminoethyl)-5-(2-naphthyl) methyloxyindole trifluoroacetate;
1-(8-quinoline)sulfonyl-3-(2-aminoethyl)-5-(2-naphthyl)methyloxyindole trifluoroacetate;
1-(2-thienyl)sulfonyl-3-(2-aminoethyl)-5-(4-biphenyl)methyloxyindole trifluoroacetate;
1-(2-chloro-4-fluoro)benzenesulfonyl-3-(2-aminoethyl)-5-(2-biphenyl)methyloxyindole trifluoroacetate;
1-(3-chloro-4-fluoro)benzenesulfonyl-3-(2-aminoethyl)-5-(2-naphthyl) methyloxyindole trifluoroacetate;
1-(8-quinoline)sulfonyl-3-(2-aminoethyl)-5-(2-biphenyl)methyloxyindole trifluoroacetate;
1-(4-methoxy)benzenesulfonyl-3-(2-aminoethyl)-5-(4-biphenyl) methyloxyindole trifluoroacetate; and
1-(2-thienyl)sulfonyl-3-(2-aminoethyl)-5-[(4-tert-butyl)phenyl] methyloxyindole trifluoroacetate;
or a pharmaceutically acceptable salt thereof.

12. A compound according to claim 5 selected from the group consisting of:
3-(2-aminoethyl)-5-(2-naphthyl)indole hydrochloride;
3-(2-aminoethyl)-5-(1-naphthyl)indole hydrochloride;
3-(2-aminoethyl)-5-(benzofuran-2-yl)indole hydrochloride;
3-(3-aminopropyl)-5-(1-naphthyl)indole trifluoroacetate;
3-(2-aminoethyl)-5-(4-dibenzofuranyl)indole hydrochloride;
3-(2-aminoethyl)-5-(2-thienyl)indole hydrochloride;
3-(2-aminoethyl)-5-(3-trifluoromethylphenyl)indole hydrochloride;
4-[3-(2-aminoethyl)indol-5-yl]benzophenone hydrochloride;
3-(2-aminoethyl)-5-(benzothiophen-2-yl)indole hydrochloride;
2-[5-(4-Methyl-naphthalen-1-yl)-1 H-indol-3-yl]-ethylamine;
6-[3-(2-Amino-ethyl)-1 H-indol-5-yl]-naphthalen-2-ol;
7-[3-(2-Amino-ethyl)-1 H-indol-5-yl]-3-hydroxy-naphthalene-2-carboxylic acid (2-methoxy-phenyl)-amide; and
2-[5-(6-Methoxy-naphthalen-2-yl)-1 H-indol-3-yl]-ethylamine;
or a pharmaceutically acceptable salt thereof.

13. A compound according to claim 6 selected from the group consisting of:
1-[(3-carbamoylphenyl)methyl]-3-(2-aminoethyl)-5-(4-dibenzofuranyl)indole hydrochloride;
1-(5-cyanopentyl)-3-(2-aminoethyl)-5-(4-dibenzofuranyl)indole hydrochloride;
1-(2-carbamoylethyl)-3-(2-aminoethyl)-5-(4-dibenzofuranyl)indole hydrochloride;
1-[(3-carboxyphenyl)methyl]-3-(2-aminoethyl)-5-(1-naphthyl)indole hydrochloride;
3-(2-aminoethyl)-5-(4-dibenzofuranyl)indole-1-pentanoic acid hydrochloride;
1-[(3-carbamoylphenyl)methyl]-3-(2-aminoethyl)-5-(4-dibenzofuranyl)indole hydrochloride;
1-(5-cyanopentyl)-3-(2-aminoethyl)-5-(4-dibenzofuranyl)indole hydrochloride;
1-(2-carbamoylethyl)-3-(2-aminoethyl)-5-(4-dibenzofuranyl)indole hydrochloride;
1-[(3-carboxyphenyl)methyl]-3-(2-aminoethyl)-5-(1-naphthyl)indole hydrochloride;
3-(2-aminoethyl)-5-(4-dibenzofuranyl)indole-1-pentanoic acid hydrochloride;
1-(carbamoylmethyl)-3-(2-aminoethyl)-5-(2-naphthyl)indole trifluoroacetate;
1-(N-methylcarbamoylmethyl)-3-(2-aminoethyl)-5-(1-naphthyl)indole trifluoroacetate;
1-[(3-carbamoylphenyl)methyl]-3-(2-aminoethyl)-5-(1-naphthyl)indole hydrochloride;
1-[(3-morpholinoylphenyl)methyl]-3-(2-aminoethyl)-5-(1-naphthyl)indole hydrochloride;
1-[(3-(3-trifluoromethylbenzyl)carbamoylphenyl)methyl]-3-(2-aminoethyl)-5-(1-naphthyl)indole hydrochloride;
1-[(4-carbamoylphenyl)methyl]-3-(2-aminoethyl)-5-(4-dibenzofuranyl)indole hydrochloride;
1-(carbamoylmethyl)-3-(2-aminoethyl)-5-(4-dibenzofuranyl)indole hydrochloride;
1-(4-carbomethoxyphenyl)methyl-3-(2-aminoethyl)-5-(4-dibenzofuranyl)indole hydrochloride;
1-(3-carbomethoxyphenyl)methyl-3-(2-aminoethyl)-5-(4-dibenzofuranyl)indole hydrochloride;
1-(2-cyanophenyl)methyl-3-(2-aminoethyl)-5-(4-dibenzofuranyl)indole hydrochloride;
1-(3-cyanophenyl)methyl-3-(2-aminoethyl)-5-(4-dibenzofuranyl)indole hydrochloride;
1-(4-acetamidophenyl)methyl-3-(2-aminoethyl)-5-(4-dibenzofuranyl)indole hydrochloride;
1-(4-cyanobutyl)-3-(2-aminoethyl)-5-(4-dibenzofuranyl)indole hydrochloride;
1-[(2-carbamoylphenyl)methyl]-3-(2-aminoethyl)-5-(4-dibenzofuranyl)indole hydrochloride;
1-ethyl-3-(2-aminoethyl)-5-(4-dibenzofuranyl)indole hydrochloride;
1-[(3-carboxyphenyl)methyl]-3-(2-aminoethyl)-5-(benzothiophen-2-yl)indole hydrochloride;
1-[(4-carboxyphenyl)methyl]-3-(2-aminoethyl)-5-(benzothiophen-2-yl)indole hydrochloride;
1-[(3-carboxyphenyl)methyl]-3-(2-aminoethyl)-5-(4-dibenzofuranyl)indole hydrochloride;
3-(2-aminoethyl)-5-(4-dibenzofuranyl)indole-1-butanoic acid hydrochloride;
1-[(N-(3-trifluoromethylphenyl)methyl)acetamido]-3-(2-aminoethyl)-5-(1-naphthyl)indole;
1-[(N-(4-pyridyl)methyl)acetamido]-3-(2-aminoethyl)-5-(1-naphthyl)indole;
1-[(N-(3-methoxyphenyl)methyl)acetamido]-3-(2-aminoethyl)-5-(1-naphthyl)indole;
1-[(N-(4-sulfonamidophenyl)methyl)acetamido]-3-(2-aminoethyl)-5-(1-naphthyl)indole;
1-[(N-benzyl)acetamido]-3-(2-aminoethyl)-5-(1-naphthyl)indole;
1-[(N-(2,5-difluorophenyl)methyl)acetamido]-3-(2-aaminoethyl)-5-(1-naphthyl)indole;
1-[(N-(2,4-dichlorophenyl)methyl)acetamido]-3-(2-aminoethyl)-5-(1-naphthyl)indole;
1-[(N-(2-benzimidazole)methyl)acetamido]-3-(2-aminoethyl)-5-(1-naphthyl)indole;
1-[N-(3-pyridyl)acetamido]-3-(2-aminoethyl)-5-(1-naphthyl)indole;
1-[N-(2-thiazole)acetamido]-3-(2-aminoethyl)-5-(1-naphthyl)indole;
1-[(N-(2-thiophene)methyl)acetamido]-3-(2-aminoethyl)-5-(1-naphthyl)indole;
1-[trans-(N-cyclopropylphenyl)acetamido]-3-(2-aminoethyl)-5-(1-naphthyl)indole;
1-[(N-(4-carboethoxy)piperidine)acetamido]-3-(2-aminoethyl)-5-(1-naphthyl)indole; and
1-[N-(3-methoxyphenyl)acetamido]-3-(2-aminoethyl)-5-(4-dibenzofuranyl)indole;
or a pharmaceutically acceptable salt thereof.

14. A compound according to claim 7 selected from the group consisting of:
3-(2-aminoethyl)-5-[(3-phenoxy)benzyloxy]indole hydrochloride;
3-(2-aminoethyl)-5-[(2-naphthyl)methyloxy]indole hydrochloride;
3-(2-aminoethyl)-5-[(2-phenyl)benzyloxylindole hydrochloride;
3-(2-aminoethyl)-5-[(4-phenyl)benzyloxy]indole hydrochloride;
1-(N-Carboxmethyl-N-methylcarbamoylmethyl)-3-(2-aminoethyl)-5-(4-dibenzofuranyl)indole trifluoroacetate;
3-[3-(2-Amino-ethyl)-5-(4-cyano-benzyloxy)-indol-1-ylmethyl]-benzoic acid trifluoroacetate;
({2-[3-(2-Amino-ethyl)-5-(3-cyano-benzyloxy)-indol-1-yl]-ethanoyl}-methyl-amino)-acetic acid;
3-[3-(2-Amino-ethyl)-5-(4-carbamoyl-benzyloxy)-indol-1-ylmethyl]-benzoic acid;
3-[3-(2-Amino-ethyl)-5-(4-carboxy-benzyloxy)-indol-1-ylmethyl]-benzoic acid;
3-[3-(2-Amino-ethyl)-5-(3-carboxy-benzyloxy)-indol-1-ylmethyl]-benzoic acid;
[3-(2-Amino-ethyl)-5-(4-carbamoyl-benzyloxy)-indol-1-yl]-acetic acid;
4-[3-(2-Amino-ethyl)-1-carboxymethyl-1H-indol-5-yloxymethyl]-benzoic acid;
[3-(2-Amino-ethyl)-5-(3-carbamoyl-benzyloxy)-indol-1-yl]-acetic acid;
3-[3-(2-Amino-ethyl)-1-carboxymethyl-1H-indol-5-yloxymethyl]-benzoic acid;
4-[3-(2-Amino-ethyl)-5-(3-carboxy-benzyloxy)-indol-1-ylmethyl]-benzoic acid;
4-[3-(2-Amino-ethyl)-1-(3-cyano-benzyl)-1H-indol-5-yloxymethyl]-benzoic acid;
5-[3-(2-Amino-ethyl)-5-(3-carbamoyl-benzyloxy)-indol-1-ylmethyl]-furan-2-carboxylic acid;
5-[3-(2-Amino-ethyl)-5-(3-cyano-benzyloxy)-indol-1-ylmethyl]-furan-2-carboxylic acid;
4-[3-(2-Amino-ethyl)-5-(4-carbamoyl-benzyloxy)-indol-1-ylmethyl]-benzoic acid;
5-[3-(2-Amino-ethyl)-5-(4-carbamoyl-benzyloxy)-indol-1-ylmethyl]-furan-2-carboxylic acid;
[3-(2-Amino-ethyl)-5-(4-cyano-benzyloxy)-indol-1-yl]-acetic acid;
5-[3-(2-Amino-ethyl)-5-(4-cyano-benzyloxy)-indol-1-ylmethyl]-furan-2-carboxylic acid;
4-[3-(2-Amino-ethyl)-5-(3-carbamoyl-benzyloxy)-indol-1-ylmethyl]-benzoic acid;
3-[3-(2-Amino-ethyl)-5-(3-carbamoyl-benzyloxy)-indol-1-ylmethyl]-benzoic acid;
5-[3-(2-Amino-ethyl)-5-(3-carbamoyl-benzyloxy)-indol-1-ylmethyl]-thiophene-2-carboxylic acid;
3-[3-(2-Amino-ethyl)-5-(3-cyano-benzyloxy)-indol-1-ylmethyl]-benzoic acid;
5-[3-(2-Amino-ethyl)-5-(3-cyano-benzyloxy)-indol-1-ylmethyl]-thiophene-2-carboxylic acid;
4-[3-(2-Amino-ethyl)-5-(biphenyl-4-ylmethoxy)-indol-1-ylmethyl]-benzoic acid;
3-[3-(2-Amino-ethyl)-5-(biphenyl-4-ylmethoxy)-indol-l-ylmethyl]-benzoic acid;
5-[3-(2-Amino-ethyl)-5-(biphenyl-4-ylmethoxy)-indol-1-ylmethyl]-furan-2-carboxylic acid;
6-[3-(2-Amino-ethyl)-5-(2-chloro-4-fluoro-benzyloxy)-indol-1-ylmethyl]-nicotinic acid;
5-[3-(2-Amino-ethyl)-5-(2-chloro-4-fluoro-benzyloxy)-indol-1-ylmethyl]-thiophene-2-carboxylic acid;
3-[3-(2-Amino-ethyl)-5-(2-chloro-4-fluoro-benzyloxy)-indol-1-ylmethyl]-benzoic acid;
5-[3-(2-Amino-ethyl)-5-(2-chloro-4-fluoro-benzyloxy)-indol-1-ylmethyl]-furan-2-carboxylic acid;
4-[3-(2-Amino-ethyl)-5-(2-chloro-4-fluoro-benzyloxy)-indol-1-ylmethyl]-benzoic acid;
(S)-1-{2-[3-(2-Amino-ethyl)-5-(2-chloro-4-fluoro-benzyloxy)-indol-1-yl]-ethanoyl}-pyrrolidine-2-carboxylic acid;
6-[3-(2-Amino-ethyl)-5-(biphenyl-2-ylmethoxy)-indol-1-ylmethyl]-nicotinic acid;
5-[3-(2-Amino-ethyl)-5-(biphenyl-2-ylmethoxy)-indol-1-ylmethyl]-thiophene-2-carboxylic acid;
3-[3-(2-Amino-ethyl)-5-(biphenyl-2-ylmethoxy)-indol-1-ylmethyl]-benzoic acid;
5-[3-(2-Amino-ethyl)-5-(biphenyl-2-ylmethoxy)-indol-1-ylmethyl]-furan-2-carboxylic acid;
4-[3-(2-Amino-ethyl)-5-(biphenyl-2-ylmethoxy)-indol-1-ylmethyl]-benzoic acid;
6-[3-(2-Amino-ethyl)-5-(2-benzyloxy-ethoxy)-indol-1-ylmethyl]-nicotinic acid;
5-[3-(2-Amino-ethyl)-5-(2-benzyloxy-ethoxy)-indol-1-ylmethyl]-thiophene-2-carboxylic acid;
3-[3-(2-Amino-ethyl)-5-(2-benzyloxy-ethoxy)-indol-1-ylmethyl]-benzoic acid;
5-[3-(2-Amino-ethyl)-5-(2-benzyloxy-ethoxy)-indol-1-ylmethyl]-furan-2-carboxylic acid;
4-[3-(2-Amino-ethyl)-5-(2-benzyloxy-ethoxy)-indol-1-ylmethyl]-benzoic acid;
3-[3-(2-Amino-ethyl)-5-(2-fluoro-benzyloxy)-indol-1-ylmethyl]-benzoic acid;
5-[3-(2-Amino-ethyl)-5-(2-fluoro-benzyloxy)-indol-1-ylmethyl]-furan-2-carboxylic acid;
4-[3-(2-Amino-ethyl)-5-(2-fluoro-benzyloxy)-indol-1-ylmethyl]-benzoic acid;
2-[3-(2-Amino-ethyl)-5-(2-fluoro-benzyloxy)-indol-1-ylmethyl]-5-bromo-benzoic acid;
5-[3-(2-Amino-ethyl)-5-(3-nitro-benzyloxy)-indol-1-ylmethyl]-thiophene-2-carboxylic acid;
3-[3-(2-Amino-ethyl)-5-(3-nitro-benzyloxy)-indol-1-ylmethyl]-benzoic acid;
5-[3-(2-Amino-ethyl)-5-(3-nitro-benzyloxy)-indol-1-ylmethyl]-furan-2-carboxylic acid;
6-[3-(2-Amino-ethyl)-5-(3-cyano-benzyloxy)-indol-1-ylmethyl]-nicotinic acid;
1-{2-[3-(2-Amino-ethyl)-5-(3-cyano-benzyloxy)-indol-1-yl]-ethanoyl}-pyrrolidine-2-carboxylic acid;
4-[3-(2-Amino-ethyl)-5-(6-chloro-benzo[1,3]dioxol-5-ylmethoxy)-indol-1-ylmethyl]-benzoic acid;
6-[3-(2-Amino-ethyl)-5-(3-methoxy-benzyloxy)-indol-1-ylmethyl]-nicotinic acid;
5-[3-(2-Amino-ethyl)-5-(3-methoxy-benzyloxy)-indol-1-ylmethyl]-furan-2-carboxylic acid;
4-[3-(2-Amino-ethyl)-5-(3-methoxy-benzyloxy)-indol-1-ylmethyl]-benzoic acid;
6-[3-(2-Amino-ethyl)-5-(6-fluoro-4H-benzo[1,3]dioxin-8-ylmethoxy)-indol-1-ylmethyl]-nicotinic acid;
5-[3-(2-Amino-ethyl)-5-(6-fluoro-4H-benzo[1,3]dioxin-8-ylmethoxy)-indol-1-ylmethyl]-thiophene-2-carboxylic acid;
3-[3-(2-Amino-ethyl)-5-(6-fluoro-4H-benzo[1,3]dioxin-8-ylmethoxy)-indol-1-ylmethyl]-benzoic acid;
5-[3-(2-Amino-ethyl)-5-(6-fluoro-4H-benzo[1,3]dioxin-8-ylmethoxy)-indol-1-ylmethyl]-furan-2-carboxylic acid;
4-[3-(2-Amino-ethyl)-5-(6-fluoro-4H-benzo[1,3]dioxin-8-ylmethoxy)-indol-1-ylmethyl]-benzoic acid;
2-[3-(2-Amino-ethyl)-5-(6-fluoro-4H-benzo[1,3]dioxin-8-ylmethoxy)-indol-1-ylmethyl]-5-bromo-benzoic acid;
1-{2-[3-(2-Amino-ethyl)-5-(6-fluoro-4H-benzo[1,3]dioxin-8-ylmethoxy)-indol-1-yl]-ethanoyl}-pyrrolidine-2-carboxylic acid;
5-[3-(2-Amino-ethyl)-5-(3-cyano-benzyloxy)-indol-1-ylmethyl]-thiophene-2-carboxylic acid;
3-[3-(2-Amino-ethyl)-5-(3-cyano-benzyloxy)-indol-1-ylmethyl]-benzoic acid;
6-[3-(2-Amino-ethyl)-5-(3-nitro-benzyloxy)-indol-1-ylmethyl]-nicotinic acid;
2-[3-(2-Amino-ethyl)-5-(3-nitro-benzyloxy)-indol-1-ylmethyl]-5-bromo-benzoic acid;
(S)-1-{2-[3-(2-Amino-ethyl)-5-benzyloxy-indol-1-yl]-ethanoyl}-pyrrolidine-2-carboxylic acid;
2-[3-(2-Amino-ethyl)-5-(3-cyano-benzyloxy)-indol-1-ylmethyl]-5-bromo-benzoic acid;
5-[3-(2-Amino-ethyl)-5-(6-chloro-benzo[1,3]dioxol-5-ylmethoxy)-indol-1-ylmethyl]-thiophene-2-carboxyic acid;
3-[3-(2-Amino-ethyl)-5-(3-methoxy-benzyloxy)-indol-1-ylmethyl]-benzoic acid;
2-[3-(2-Amino-ethyl)-5-(6-chloro-benzo [1,3]dioxol-5-ylmethoxy)-indol-1-ylmethyl]-5-bromo-benzoic acid;
5-[3-(2-Amino-ethyl)-5-benzyloxy-indol-1-ylmethyl]-furan-2-carboxylic acid;
5-[3-(2-Amino-ethyl)-5-(3-carbamoyl-benzyloxy)-indol-1-ylmethyl]-2-bromo-benzoic acid;
3-[3-(2-Amino-ethyl)-5-(6-chloro-benzo[1,3]dioxol-5-ylmethoxy)-indol-1-ylmethyl]-benzoic acid;
5-[3-(2-Amino-ethyl)-5-(6-chloro-benzo[1,3]dioxol-5-ylmethoxy)-indol-1-ylmethyl]-furan-2-carboxylic acid;
or a pharmcaeutically acceptable salt thereof.

15. A method of treating or preventing infection which comprises administering to a subject in need thereof, an effective amount of a compound according to claim 1.

16. A method according to claim 15 which involves inhibiting a virus.

17. A method according to claim 16 which involves inhibiting a virus selected from the group consisting of a herpesvirus, a betaherpesvirus, and a cytomegalovirus.

18. A method according to claim 17 which involves inhibiting a human cytomegalovirus.

19. A method according to claim 15 which involves inhibition of the interaction between the major capsid protein and either the full length protease or the scaffolding proteins.

20. A method according to claim 15 in which the compound is administered in an oral dosage form.
